(19) United States
(12) Patent Application Publication
(10) Pub. No.: US 2013/0190327 A1
(43) Pub. Date: Jul. 25, 2013

(54) BIS-FATTY ACID CONJUGATES AND THEIR USES

(71) Applicants: Jill C. Milne, Brookline, MA (US); Michael R. Jirousek, Cambridge, MA (US); Jean E. Bemis, Arlington, MA (US); Chi B. Vu, Arlington, MA (US)

(72) Inventors: Jill C. Milne, Brookline, MA (US); Michael R. Jirousek, Cambridge, MA (US); Jean E. Bemis, Arlington, MA (US); Chi B. Vu, Arlington, MA (US)

(73) Assignee: CATABASIS PHARMACEUTICALS INC, CAMBRIDGE, MA (US)

(21) Appl. No.: 13/581,392

(22) PCT Filed: Feb. 25, 2011
(86) PCT No.: PCT/US11/26305

§ 371 Date: Nov. 12, 2012

Related U.S. Application Data
(60) Provisional application No. 61/308,666, filed on Feb. 26, 2010.
(60) Provisional application No. 61/310,955, filed on Mar. 5, 2010.

(51) Int. Cl. C07C 233/49; C07D 295/195; C07D 339/04

(52) U.S. Cl. CPC C07C 233/49; C07D 339/04; C07D 295/195
USPC 514/255.01; 554/56; 549/39; 554/58; 544/387; 514/616; 514/440; 514/547; 514/560

(57) ABSTRACT

The invention relates to bis-fatty acid conjugates; compositions comprising an effective amount of a bis-fatty acid conjugate; and methods for treating or preventing cancer, a metabolic disease or a neurodegenerative disease comprising the administration of an effective amount of a bis-fatty acid conjugate.

### PRIORITY

The present application claims the benefit of U.S. Provisional Application No. 61/308,666 filed Feb. 26, 2010, and U.S. Provisional Application No. 61/310,955 filed Mar. 5, 2010. The entire disclosures of those applications are relied on for all purposes and are incorporated into this application by reference.

### FIELD OF THE INVENTION

The invention relates to bis-fatty acid conjugates; compositions comprising an effective amount of a bis-fatty acid conjugate; and methods for treating or preventing a given type of cancer, a metabolic, autoimmune or neurodegenerative disorder comprising the administration of an effective amount of a bis-fatty acid conjugate. All patents, patent applications, and publications cited herein are hereby incorporated by reference in their entireties.

### BACKGROUND OF THE INVENTION

Oily cold water fish, such as salmon, trout, herring, and tuna are the source of dietary marine omega-3 fatty acids, with eicosapentaenoic acid (EPA) and docosahexaenoic acid (DHA) being the key marine derived omega-3 fatty acids. Omega-3 fatty acids have previously been shown to improve insulin sensitivity and glucose tolerance in normoglycemic men and in obese individuals. Omega-3 fatty acids have also been shown to improve insulin resistance in obese and non-obese patients with an inflammatory phenotype. Lipid, glucose, and insulin metabolism have been shown to improve in overweight hypertensive subjects through treatment with omega-3 fatty acids. Omega-3 fatty acids (EPA/DHA) have also been shown to decrease triglycerides and to reduce the risk for sudden death caused by cardiac arrhythmias in addition to improve mortality in patients at risk of a cardiovascular event. Omega-3 fatty acids have also been taken as dietary supplements part of therapy used to treat dyslipidemia, and anti-inflammatory properties. A higher intake of omega-3 fatty acids lower levels of circulating TNF-α and IL-6, two of the cytokines that are markedly increased during inflammation processes (Chapkin et al, Prostaglandins, Leukot Essent Fatty Acids 2009, 81, p. 187-191; Duda et al, Cardiovasc Res 2009, 84, p. 33-41). In addition, a higher intake of omega-3 fatty acids has been shown to increase levels of the well-characterized anti-inflammatory cytokine IL-10 (Bradley et al, Obesity (Silver Spring) 2008, 16, p. 938-944). A recent study (Wang et al, Molecular Pharmaceutics 2010, 7, p. 2185-2193) has demonstrated that DHA could also induce the Nrf2 and the Nrf2-target gene Heme-oxygenase 1 (HO-1) and this pathway could play a significant role in suppressing LPS-mediated inflammation. A number of studies have now indicated that DHA could play a significant role in cancer (For reviews see: Gleissman, H. et al Experimental Cell Research 2010, 316, p. 1365-73; Bougnoux, P. et al Progress in Lipid Research 2010, 49, p. 76-86; Spencer, L. et al, Eur. J. Cancer 2009, 45, p. 2077-86; Serini, S. et al Apoptosis 2009, 14, p. 135-152; Browever, I. A. Prostaglandins, Leukotrienes and Essential Fatty Acids 2008, 79, p. 97-99). For instance, DHA was able to induce p53-dependent growth inhibition of transformed colon and lung carcinomas (Kikawa et al, J. of Cancer Science and Therapy, 2011, 3, p. 1-4). DHA has also been shown to prevent breast cancer cell metastasis to bone in a mouse model utilizing MDA-MB-231 human breast cancer cells (Mandal et al, Biochem. & Biophys. Res. Communications 2010, 402, p. 602-607).

Both DHA and EPA are characterized as long chain fatty acids (aliphatic portion between 12-22 carbons). Medium chain fatty acids are characterized as those having the aliphatic portion between 6-12 carbons. Lipoic acid is a medium chain fatty acid found naturally in the body. It plays many important roles such as free radical scavenger, chelator to heavy metals and signal transduction mediator in various inflammatory and metabolic pathways, including the NF-KB pathway (Shay, K. P. et al. Biochim. Biophys. Acta 2009, 1790, 1149-1160). Lipoic acid has been found to be useful in a number of chronic diseases that are associated with oxidative stress (for a review see Smith, A. R. et al Curr. Med. Chem. 2004, 11, p. 1135-46). Lipoic acid has now been evaluated in the clinic for the treatment of diabetes (Morcos, M. et al Diabetes Res. Clin. Pract. 2001, 52, p. 175-183) and diabetic neuropathy (Mijnhout, G. S. et al Neth. J. Med. 2010, 110, p. 158-162). Lipoic acid has also been found to be potentially useful in treating cardiovascular diseases (Ghibu, S. et al, J. Cardiovasc. Pharmacol. 2009, 54, p. 391-8), Alzheimer's disease (Maczurek, A. et al, Adv. Drug Deliv. Rev. 2008, 60, p. 1463-70) and multiple sclerosis (Yadav, V. Multiple Sclerosis 2005, 11, p. 159-65; Salinthone, S. et al, Endocr. Metab. Immune Disord. Drug Targets 2008, 8, p. 132-42).

Chronic oxidative stress and inflammation have now been linked to the development and progression of a number of debilitating diseases. Some of these diseases include renal failure, heart failure, atherosclerosis, osteoporosis, cancer, chronic obstructive pulmonary disease (COPD), Parkinson's disease and Alzheimer's disease. Activation of the Nrf2 pathway in order to resolve this chronic oxidative stress and inflammation appears to be a particularly promising new therapeutic approach (For a review see Gozzelino, R. et al Annu. Rev. Pharmacol. Toxicol. 2010, 50, p. 323-54). For instance, small molecule activators of Nrf2 have now been shown to be effective in the cisplatin-induced nephrotoxicity mouse model (Aleksunes et al, J. Pharmacology & Experimental Therapeutics 2010, 335, p. 2-12), the transgenic Tg19959 mouse model of Alzheimer's disease (Dumont et al, J. Neurochem. 2009, 109, p. 502-12), the mouse model for COPD (Sussan, T. E. et al Proc. Natl. Acad. Sci. USA 2009, 106, p. 250-5), and the murine 4T1 breast tumor model (Ling, X. et al Cancer Res. 2007, 67, p. 4210-8).

The ability to provide the effects of fatty acids in a synergistic way would provide benefits in treating a variety of cancer, metabolic, autoimmune and neurodegenerative diseases.

### SUMMARY OF THE INVENTION

The invention is based in part on the discovery of bis-fatty acid conjugates and their demonstrated effects in achieving improved treatment that cannot be achieved by administering fatty acids alone or in combination. These novel conjugates are useful in the treatment and prevention of diseases and disorders associated with inflammation. For example, the conjugates described herein are useful in the treatment or prevention of metabolic disorders including atherosclerosis, dyslipidemia, coronary heart disease, hypertriglyceridemia, hypercholesterimia, Type 2 diabetes, elevated cholesterol, metabolic syndrome, diabetic nephropathy, IgA nephropathy, chronic kidney disease (CKD) and cardiovascular disease. In addition, they are useful in the treatment of autoimmune diseases such as rheumatoid arthritis, psoriasis, systemic lupus erythematosus, inflammatory bowel diseases such as, but not limited to colitis and Crohn's disease, respiratory diseases such as, but not limited to, asthma, cystic fibrosis, COPD, and neurodegenerative diseases such as multiple sclerosis, Parkinson's disease, Alzheimer's disease, Huntington's disease, amyotrophic lateral sclerosis (ALS) and muscular dystrophy. The compounds described herein are also useful in treating a variety of cancer such as carcinoma, sarcoma, lymphoma, leukemia, melanoma, mesothelioma, multiople myeloma, seminoma, and cancer of the bladder, blood, bone, brain, breast, central nervous system, colon, endometrium, esophagus, genitourinary tract, head, larynx, liver, lung, neck, ovary, pancreas, prostate, testicle, spleen, small intestine, large intestine or stomach.

Accordingly in one aspect, a molecular conjugate is described which comprises two or more fatty acids covalently linked wherein the linker comprises at least one amide, wherein the fatty acids are selected from the group consisting of omega-3 fatty acids, fatty acids that are metabolized in vivo to omega-3 fatty acids, and lipoic acid, and the conjugate is capable of hydrolysis to produce free fatty acids, with the proviso that the molecular conjugate is not (4Z,7Z,10Z,13Z,16Z,19Z)-docosa-4,7,10,13,16,19-hexaenoic acid {2-[2-((4Z,7Z,10Z,13Z,16Z,19Z)-docosa-4,7,10,13,16,19-hexaenoylamino)-ethylamino]-ethyl}-amide (A); (5Z,8Z,11Z,14Z,17Z)-icosa-5,8,11,14,17-pentaenoic acid {2-[2-((5Z,8Z,11Z,14Z,17Z)-icosa-5,8,11,14,17-pentaenoylamino)-ethylamino]-ethyl}-amide (B); (4Z,7Z,10Z,13Z,16Z,19Z)-docosa-4,7,10,13,16,19-hexaenoic acid {2-[2-((4Z,7Z,10Z,13Z,16Z,19Z)-docosa-4,7,10,13,16,19-hexaenoylamino)-ethoxy]-ethyl}-amide (C); (5Z,8Z,11Z,14Z,17Z)-icosa-5,8,11,14,17-pentaenoic acid {2-[2-((5Z,8Z,11Z,14Z,17Z)-icosa-5,8,11,14,17-pentaenoylamino)-ethoxy]-ethyl}-amide (D); or (S)-2,6-Bis-((4Z,7Z,10Z,13Z,16Z,19Z)-docosa-4,7,10,13,16,19-hexaenoylamino)-hexanoic acid (E). In some embodiments, the fatty acid is selected from the group consisting of all-cis-7,10,13-hexadecatrienoic acid, α-linolenic acid, stearidonic acid, eicosatrienoic acid, eicosatetraenoic acid, eicosapentaenoic acid (EPA), docosapentaenoic acid, docosahexaenoic acid (DHA), tetracosapentaenoic acid, tetracosahexaenoic acid and lipoic acid. In other embodiments, the fatty acid is selected from eicosapentaenoic acid, docosahexaenoic acid and lipoic acid. In some embodiments, the hydrolysis is enzymatic. In some embodiments, a pharmaceutical composition is described comprising a covalently linked molecular conjugate described herein and a pharmaceuictally accetable carrier. In some embodiments, the pharmaceutical composition comprises at least one compound selected from (4Z,7Z,10Z,13Z,16Z,19Z)-docosa-4,7,10,13,16,19-hexaenoic acid {2-[2-((4Z,7Z,10Z,13Z,16Z,19Z)-docosa-4,7,10,13,16,19-hexaenoylamino)-ethylamino]-ethyl}-amide (A); (5Z,8Z,11Z,14Z,17Z)-icosa-5,8,11,14,17-pentaenoic acid {2-[2-((5Z,8Z,11Z,14Z,17Z)-icosa-5,8,11,14,17-pentaenoylamino)-ethylamino]-ethyl}-amide (B); (4Z,7Z,10Z,13Z,16Z,19Z)-docosa-4,7,10,13,16,19-hexaenoic acid {2-[2-((4Z,7Z,10Z,13Z,16Z,19Z)-docosa-4,7,10,13,16,19-hexaenoylamino)-ethoxy]-ethyl}-amide (C); (5Z,8Z,11Z,14Z,17Z)-icosa-5,8,11,14,17-pentaenoic acid {2-[2-((5Z,8Z,11Z,14Z,17Z)-icosa-5,8,11,14,17-pentaenoylamino)-ethoxy]-ethyl}-amide (D); or (S)-2,6-Bis-((4Z,7Z,10Z,13Z,16Z,19Z)-docosa-4,7,10,13,16,19-hexaenoylamino)-hexanoic acid (E) and a pharmaceutically accetable carrier.

In another aspect, compounds of the Formula I are described:

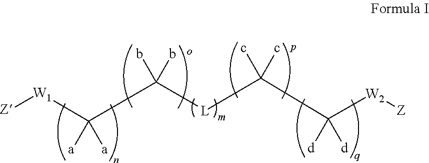

and pharmaceutically acceptable salts, hydrates, solvates, prodrugs, enantiomers and stereoisomers thereof;

wherein

W₁ and W₂ are each independently O, S, NH, NR, or W₁ and W₂ can be taken together can form an imidazolidine or piperazine group;

with the proviso that W₁ and W₂ can not simultaneously be O and one of W1 and W2 is NH or NR;

each a, b, c, and d is independently —H, -D, —CH₃, —OCH₃, —OCH₂CH₃, —C(O)OR, —O—Z, or benzyl, or two of a, b, c, and d can be taken together, along with the single carbon to which they are bound, to form a cycloalkyl or heterocycle;

each n, o, p, and q is independently 0, 1 or 2;

L is independently null, —O—, —S—, —S(O)—, —S(O)₂—, —S—S—, —(C₁-C₆alkyl)-, —(C₃-C₆cycloalkyl)-, a heterocycle, a heteroaryl,

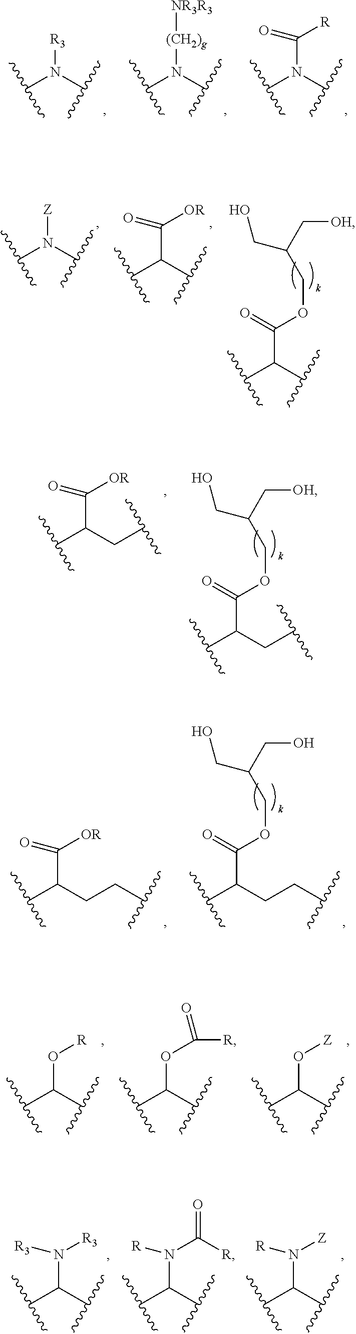

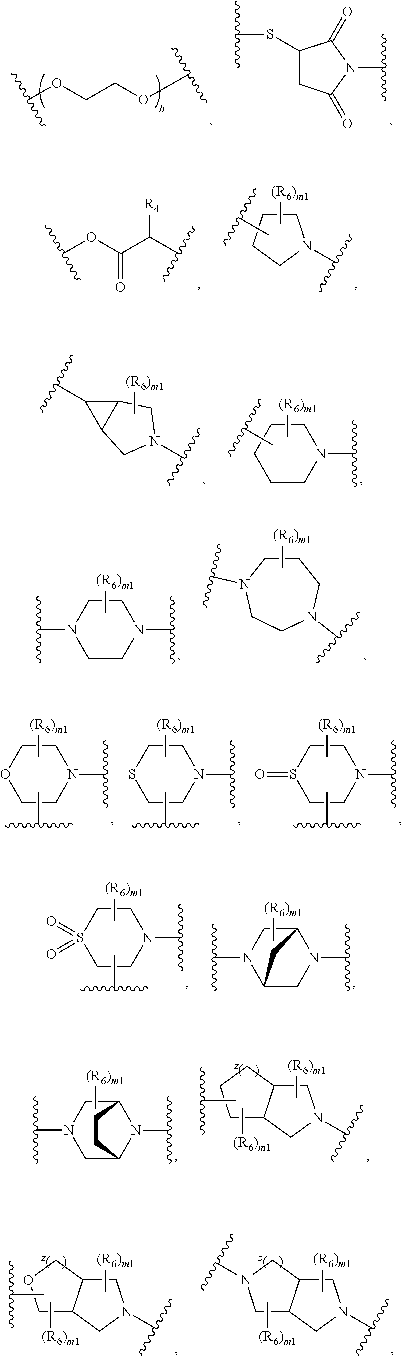

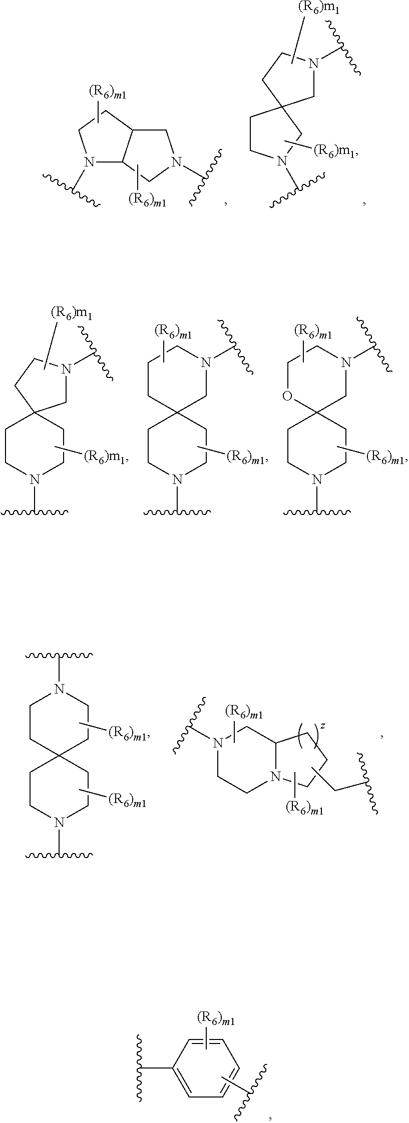

wherein the representation of L is not limited directionally left to right as is depicted, rather either the left side or the right side of L can be bound to the W₁ side of the compound of Formula I;

R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

each g is independently 2, 3 or 4;

each h is independently 1, 2, 3 or 4;

m is 0, 1, 2, or 3; if m is more than 1, then L can be the same or different;

m1 is 0, 1, 2 or 3;

k is 0, 1, 2, or 3;

z is 1, 2, or 3;

each R₃ is independently H or C₁-C₆ alkyl that can be optionally substituted with either O or N and in NR₃R₃, both R₃ when taken together with the nitrogen to which they are attached can form a heterocyclic ring such as a pyrrolidine, piperidine, morpholine, piperazine or pyrrole;

each R₄ independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z and Z′ is independently —H,

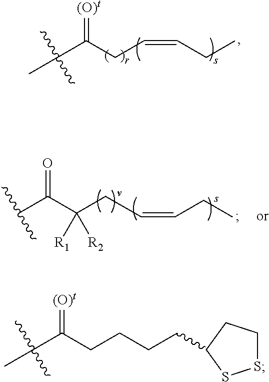

with the proviso that there is at least two of

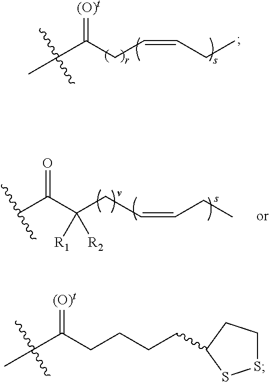

in the compound;

each r is independently 2, 3, or 7;

each s is independently 3, 5, or 6;

each t is independently 0 or 1;

each v is independently 1, 2, or 6;

R₁ and R₂ are independently —H, -D, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C₁-C₃ alkyl, or straight or branched C₁-C₄ alkyl optionally substituted with OH, or halogen;

with the further proviso that the compound is not
(4Z,7Z,10Z,13Z,16Z,19Z)-docosa-4,7,10,13,16,19-hexaenoic acid {2-[2-((4Z,7Z,10Z,13Z,16Z,19Z)-docosa-4,7,10,13,16,19-hexaenoylamino)-ethylamino]-ethyl}-amide (A); (5Z,8Z,11Z,14Z,17Z)-icosa-5,8,11,14,17-pentaenoic acid {2-[2-((5Z,8Z,11Z,14Z,17Z)-icosa-5,8,11,14,17-p entaenoylamino)-ethylamino]-ethyl}-amide (B); (4Z,7Z,10Z,13Z,16Z,19Z)-docosa-4,7,10,13,16,19-hexaenoic acid {2-[2-((4Z,7Z,10Z,13Z,16Z,19Z)-docosa-4,7,10,13,16,19-hexaenoylamino)-ethoxy]-ethyl}-amide (C); (5Z,8Z,11Z,14Z,17Z)-icosa-5,8,11,14,17-pentaenoic acid {2-[2-((5Z,8Z,11Z,14Z,17Z)-icosa-5,8,11,14,17-pentaenoylamino)-ethoxy]-ethyl}-amide (D); or (S)-2,6-Bis-((4Z,7Z,10Z,13Z,16Z,19Z)-docosa-4,7,10,13,16,19-hexaenoylamino)-hexanoic acid (E)

In Formula I, any one or more of H may be substituted with a deuterium. It is also understood in Formula I that a methyl substituent can be substituted with a C₁-C₆ alkyl.

Also described are pharmaceutical formulations comprising at least one bis-fatty acid conjugate of the Formula I′

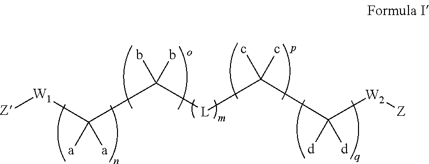

and pharmaceutically acceptable salts, hydrates, solvates, prodrugs, enantiomers and stereoisomers thereof;

wherein

W₁ and W₂ are each independently O, S, NH, NR, or W₁ and W₂ can be taken together can form an imidazolidine or piperazine group;

with the proviso that W₁ and W₂ can not simultaneously be 0 and one of W1 and W2 is NH or NR;

each a, b, c, and d is independently —H, -D, —CH₃, —OCH₃, —OCH₂CH₃, —C(O)OR, —O—Z, or benzyl, or two of a, b, c, and d can be taken together, along with the single carbon to which they are bound, to form a cycloalkyl or heterocycle;

each n, o, p, and q is independently 0, 1 or 2;

L is independently null, —O—, —S—, —S(O)—, —S(O)₂—, —S—S—, —(C₁-C₆alkyl)-, —(C₃-C₆cycloalkyl)-, a heterocycle, a heteroaryl,

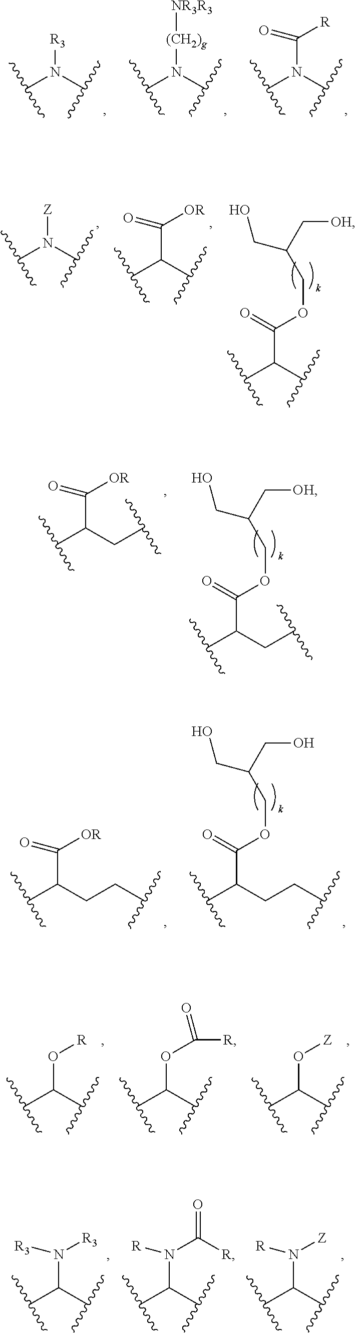

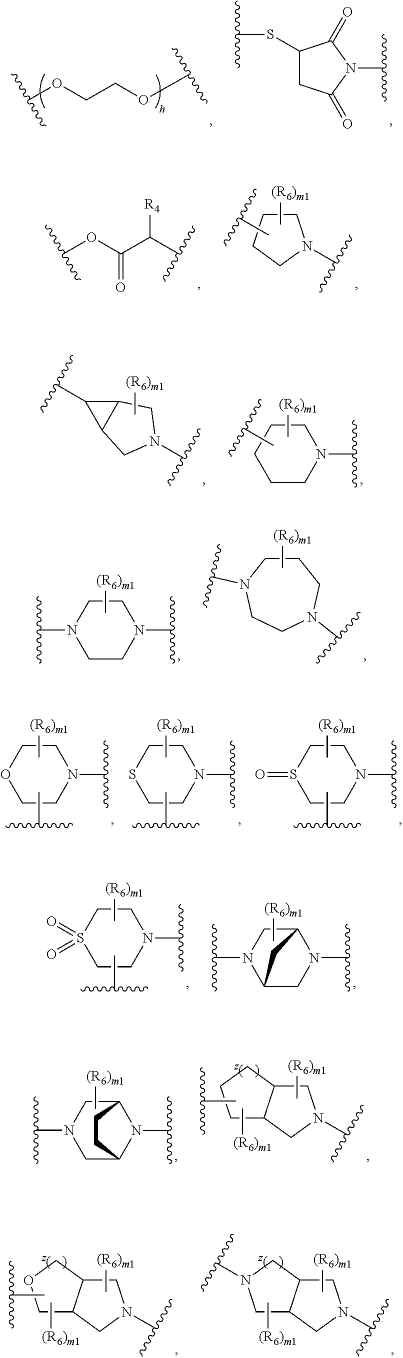

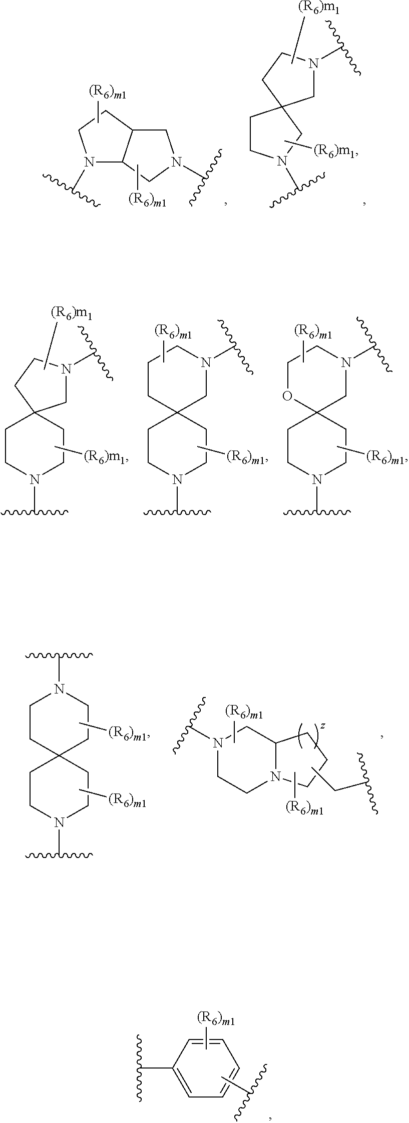

wherein the representation of L is not limited directionally left to right as is depicted, rather either the left side or the right side of L can be bound to the W₁ side of the compound of Formula I′;

R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

each g is independently 2, 3 or 4;

each h is independently 1, 2, 3 or 4;

m is 0, 1, 2, or 3; if m is more than 1, then L can be the same or different;

m1 is 0, 1, 2 or 3;

k is 0, 1, 2, or 3;

z is 1, 2, or 3;

each R₃ is independently H or C₁-C₆ alkyl that can be optionally substituted with either O or N and in NR₃R₃, both R₃ when taken together with the nitrogen to which they are attached can form a heterocyclic ring such as a pyrrolidine, piperidine, morpholine, piperazine or pyrrole;

each R₄ independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z and Z′ is independently —H,

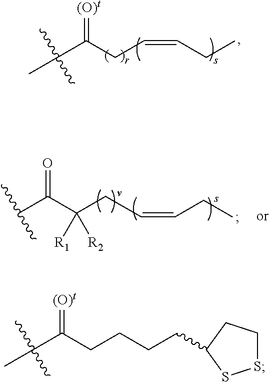

with the proviso that there is at least two of

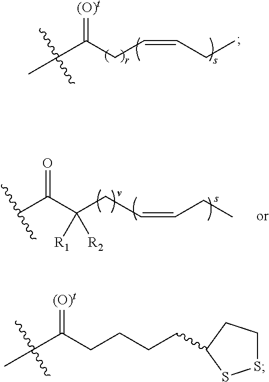

in the compound;

each r is independently 2, 3, or 7;

each s is independently 3, 5, or 6;

each t is independently 0 or 1;

each v is independently 1, 2, or 6;

R₁ and R₂ are independently —H, -D, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C₁-C₃ alkyl, or straight or branched C₁-C₄ alkyl optionally substituted with OH, or halogen.

Also described herein are methods of treating a disease susceptible to treatment with a bis-fatty acid conjugate in a patient in need thereof by administering to the patient an effective amount of a bis-fatty acid conjugate.

Also described herein are methods of treating metabolic diseases, autoimmune disease, inflammatory diseases, respiratory disease, or neurodegenerative diseases by administering to a patient in need thereof an effective amount of a bis-fatty acid conjugate or a pharmaceutical composition thereof.

The invention also includes pharmaceutical compositions that comprise an effective amount of a bis-fatty acid conjugate and a pharmaceutically acceptable carrier. The compositions are useful for treating or preventing a metabolic disease. The invention includes a bis-fatty acid conjugate provided as a pharmaceutically acceptable prodrug, a hydrate, a salt, such as a pharmaceutically acceptable salt, enantiomer, stereoisomer, or mixtures thereof.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### BRIEF DESCRIPTION OF THE FIGURES

FIG. 1 depicts the effects of compound I-66 in the ApoB secretion assay.

FIG. 2 depicts the effect of compound I-98 on IL-113.

FIG. 3 depicts the effect of compound I-37 on Hmox-1.

FIG. 4 depicts the effect of compound I-67 on Hmox-1.

### DETAILED DESCRIPTION OF THE INVENTION

Metabolic disorders are a wide variety of medical disorders that interfere with a subject's metabolism. Metabolism is the process a subject's body uses to transform food into energy. Metabolism in a subject with a metabolic disorder is disrupted in some way. Autoimmune diseases arise from an overactive immune response of the body against tissues normally present in the body. Neurodegenerative diseases result from the deterioration of neurons or their myelin sheaths, which would eventually lead to a variety of CNS-related dysfunctions. The bis-fatty acid conjugates possess the ability to treat or prevent metabolic disorders, autoimmune diseases, inflammatory bowel diseases, respiratory diseases, or neurodegenerative diseases. In addition, the bis-fatty acid conjugates can also be used to treat a variety of cancer such as such as carcinoma, sarcoma, lymphoma, leukemia, melanoma, mesothelioma, multiople myeloma, seminoma, and cancer of the bladder, blood, bone, brain, breast, central nervous system, colon, endometrium, esophagus, genitourinary tract, head, larynx, liver, lung, neck, ovary, pancreas, prostate, testicle, spleen, small intestine, large intestine or stomach.

The bis-fatty acid conjugates have been designed to bring together fatty acids into a single covalently linked molecular conjugate. The activity of the bis-fatty acid conjugates is substantially greater than the sum of the individual components of the molecular conjugate, suggesting that the activity induced by the bis-fatty acid conjugates is synergistic.

### DEFINITIONS

The following definitions are used in connection with the bis-fatty acid conjugates:

The term “bis-fatty acid conjugates” includes any and all possible isomers, stereoisomers, enantiomers, diastereomers, tautomers, pharmaceutically acceptable salts, hydrates, solvates, and prodrugs of the bis-fatty acid conjugates described herein.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

Unless otherwise specifically defined, the term “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. The substituents can themselves be optionally substituted.

“C₁-C₃ alkyl” refers to a straight or branched chain saturated hydrocarbon containing 1-3 carbon atoms. Examples of a C₁-C₃ alkyl group include, but are not limited to, methyl, ethyl, propyl and isopropyl.

“C₁-C₄ alkyl” refers to a straight or branched chain saturated hydrocarbon containing 1-4 carbon atoms. Examples of a C₁-C₄ alkyl group include, but are not limited to, methyl, ethyl, propyl, butyl, isopropyl, isobutyl, sec-butyl and tert-butyl.

“C₁-C₅ alkyl” refers to a straight or branched chain saturated hydrocarbon containing 1-5 carbon atoms. Examples of a C₁-C₅ alkyl group include, but are not limited to, methyl, ethyl, propyl, butyl, pentyl, isopropyl, isobutyl, sec-butyl and tert-butyl, isopentyl and neopentyl.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, and neopentyl.

The term “cycloalkyl” refers to a cyclic hydrocarbon containing 3-6 carbon atoms. Examples of a cycloalkyl group include, but are not limited to, cyclopropyl, cyclobutyl, cyclopentyl and cyclohexyl. It is understood that any of the substitutable hydrogens on a cycloalkyl can be substituted with halogen, C₁-C₃ alkyl, hydroxyl, alkoxy and cyano groups.

The term “heterocycle” as used herein refers to a cyclic hydrocarbon containing 3-6 atoms wherein at least one of the atoms is an O, N, or S. Examples of heterocycles include, but are not limited to, aziridine, oxirane, thiirane, azetidine, oxetane, thietane, pyrrolidine, tetrahydrofuran, tetrahydrothiophene, piperidine, tetrahydropyran, thiane, imidazolidine, oxazolidine, thiazolidine, dioxolane, dithiolane, piperazine, oxazine, dithiane and dioxane.

The term “heteroaryl” as used herein refers to a monocyclic or bicyclic ring structure having 5 to 12 ring atoms wherein one or more of the ring atoms is a heteroatom, e.g. N, O or S and wherein one or more rings of the bicyclic ring structure is aromatic. Some examples of heteroaryl are pyridyl, furyl, pyrrolyl, thienyl, thiazolyl, oxazolyl, imidazolyl, indolyl, tetrazolyl, benzofuryl, xanthenes and dihydroindole. It is understood that any of the substitutable hydrogens on a heteroaryl can be substituted with halogen, C₁-C₃ alkyl, hydroxyl, alkoxy and cyano groups.

The term “any one of the side chains of the naturally occurring amino acids” as used herein means a side chain of any one of the following amino acids: Isoleucine, Alanine, Leucine, Asparagine, Lysine, Aspartate, Methionine, Cysteine, Phenylalanine, Glutamate, Threonine, Glutamine, Tryptophan, Glycine, Valine, Proline, Arginine, Serine, Histidine and Tyrosine.

The term “fatty acid” as used herein means an omega-3 fatty acid and fatty acids that are metabolized in vivo to omega-3 fatty acids. Non-limiting examples of fatty acids are all-cis-7,10,13-hexadecatrienoic acid, α-linolenic acid (ALA or all-cis-9,12,15-octadecatrienoic acid), stearidonic acid (STD or all-cis-6,9,12,15-octadecatetraenoic acid), eicosatrienoic acid (ETE or all-cis-11,14,17-eicosatrienoic acid), eicosatetraenoic acid (ETA or all-cis-8,11,14,17-eicosatetraenoic acid), eicosapentaenoic acid (EPA or all-cis-5,8,11,14,17-eicosapentaenoic acid), docosapentaenoic acid (DPA, clupanodonic acid or all-cis-7,10,13,16,19-docosapentaenoic acid), docosahexaenoic acid (DHA or all-cis-4,7,10,13,16,19-docosahexaenoic acid), tetracosapentaenoic acid (all-cis-9,12,15,18,21-docosahexaenoic acid), or tetracosahexaenoic acid (nisinic acid or all-cis-6,9,12,15,18,21-tetracosenoic acid). In addition, the term “fatty acid” can also refer to medium chain fatty acids such as lipoic acid.

A “subject” is a mammal, e.g., a human, mouse, rat, guinea pig, dog, cat, horse, cow, pig, or non-human primate, such as a monkey, chimpanzee, baboon or rhesus, and the terms “subject” and “patient” are used interchangeably herein.

The invention also includes pharmaceutical compositions comprising an effective amount of a bis-fatty acid conjugate and a pharmaceutically acceptable carrier. The invention includes a bis-fatty acid conjugate provided as a pharmaceutically acceptable prodrug, hydrate, salt, such as a pharmaceutically acceptable salt, enantiomers, stereoisomers, or mixtures thereof.

Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “metabolic disease” as used herein refers to disorders, diseases and syndromes involving dyslipidemia, and the terms metabolic disorder, metabolic disease, and metabolic syndrome are used interchangeably herein.

An “effective amount” when used in connection with a bis-fatty acid conjugate is an amount effective for treating or preventing a metabolic disease.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The term “treating”, with regard to a subject, refers to improving at least one symptom of the subject's disorder. Treating can be curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a bis-fatty acid conjugate.

The following abbreviations are used herein and have the indicated definitions: Boc and BOC are tert-butoxycarbonyl, Boc₂O is di-tert-butyl dicarbonate, CDI is 1,1′-carbonyldiimidazole, DCC is N,N′-dicyclohexylcarbodiimide, DIEA is N,N-diisopropylethylamine, DMAP is 4-dimethylaminopyridine, DOSS is sodium dioctyl sulfosuccinate, EDC and EDCI are 1-ethyl-3-(3-dimethylaminopropyl)carbodiimide hydrochloride, EtOAc is ethyl acetate, h is hour, HATU is 2-(7-aza-1H-benzotriazole-1-yl)-1,1,3,3-tetramethyluronium hexafluorophosphate, HPMC is hydroxypropyl methylcellulose, oxone is potassium peroxymonosulfate, Pd/C is palladium on carbon, TFA is trifluoroacetic acid, TGPS is tocopherol propylene glycol succinate, THF is tetrahydrofuran, and TNF is tumor necrosis factor.

### Compounds

Accordingly in one aspect, a molecular conjugate is described which comprises two or more fatty acids covalently linked, wherein the fatty acids are selected from the group consisting of omega-3 fatty acids, fatty acids that are metabolized in vivo to omega-3 fatty acids and lipoic acid, and the conjugate is capable of hydrolysis to produce free fatty acids, with the proviso that the molecular conjugate does not encompass, (4Z,7Z,10Z,13Z,16Z,19Z)-docosa-4,7,10,13,16,19-hexaenoic acid {2-[2-((4Z,7Z,10Z,13Z,16Z,19Z)-docosa-4,7,10,13,16,19-hexaenoylamino)-ethylamino]-ethyl}-amide (A); (5Z,8Z,11Z,14Z,17Z)-icosa-5,8,11,14,17-pentaenoic acid {2-[2-((5Z,8Z,11Z,14Z,17Z)-icosa-5,8,11,14,17-pentaenoylamino)-ethylamino]-ethyl}-amide (B); (4Z,7Z,10Z,13Z,16Z,19Z)-docosa-4,7,10,13,16,19-hexaenoic acid {2-[2-((4Z,7Z,10Z,13Z,16Z,19Z)-docosa-4,7,10,13,16,19-hexaenoylamino)-ethoxy]-ethyl}-amide (C); (5Z,8Z,11Z,14Z,17Z)-icosa-5,8,11,14,17-pentaenoic acid {2-[2-((5Z,8Z,11Z,14Z,17Z)-icosa-5,8,11,14,17-pentaenoylamino)-ethoxy]-ethyl}-amide (D); or (S)-2,6-Bis-((4Z,7Z,10Z,13Z,16Z,19Z)-docosa-4,7,10,13,16,19-hexaenoylamino)-hexanoic acid (E).

In some embodiments, the fatty acids are selected from the group consisting of all-cis-7,10,13-hexadecatrienoic acid, α-linolenic acid, stearidonic acid, eicosatrienoic acid, eicosatetraenoic acid, eicosapentaenoic acid (EPA), docosapentaenoic acid, docosahexaenoic acid (DHA), tetracosapentaenoic acid, tetracosahexaenoic acid, and lipoic acid. In other embodiments, the fatty acid is selected from eicosapentaenoic acid, docosahexaenoic acid, and lipoic acid. In other embodiments, the fatty acid is selected from eicosapentaenoic acid and docosahexaenoic acid, In some embodiments, the hydrolysis is enzymatic.

In another aspect, the present invention provides bis-fatty acid conjugates according to Formula I:

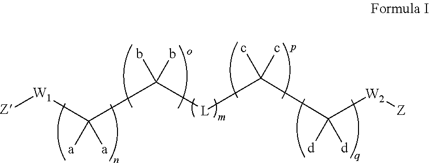

and pharmaceutically acceptable salts, hydrates, solvates, prodrugs, enantiomers and stereoisomers thereof;

wherein

W₁, W₂, a, c, b, d, e, k, m, ml, n, o, p, q, L, Z, Z′, r, s, t, v, z, R₁, R₂, R₃, R₄, R and R₆ are as defined above for Formula I,

with the proviso that there is at least two of

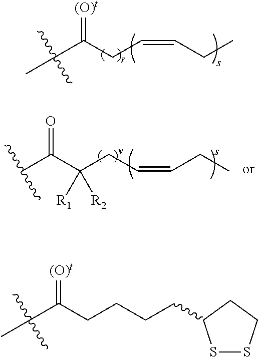

in the compound;

and with the further proviso that the compound is not
(4Z,7Z,10Z,13Z,16Z,19Z)-docosa-4,7,10,13,16,19-hexaenoic acid {2-[2-((4Z,7Z,10Z,13Z,16Z,19Z)-docosa-4,7,10,13,16,19-hexaenoylamino)-ethylamino]-ethyl}-amide (A); (5Z,8Z,11Z,14Z,17Z)-icosa-5,8,11,14,17-pentaenoic acid {2-[2-((5Z,8Z,11Z,14Z,17Z)-icosa-5,8,11,14,17-pentaenoylamino)-ethylamino]-ethyl}-amide (B); (4Z,7Z,10Z,13Z,16Z,19Z)-docosa-4,7,10,13,16,19-hexaenoic acid {2-[2-((4Z,7Z,10Z,13Z,16Z,19Z)-docosa-4,7,10,13,16,19-hexaenoylamino)-ethoxy]-ethyl}-amide (C); (5Z,8Z,11Z,14Z,17Z)-icosa-5,8,11,14,17-pentaenoic acid {2-[2-((5Z,8Z,11Z,14Z,17Z)-icosa-5,8,11,14,17-pentaenoylamino)-ethoxy]-ethyl}-amide (D); or (S)-2,6-Bis-((4Z,7Z,10Z,13Z,16Z,19Z)-docosa-4,7,10,13,16,19-hexaenoylamino)-hexanoic acid (E).

In some embodiments, one Z is

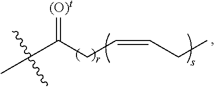

and r is 2.

In some embodiments, one Z is

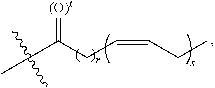

and r is 3.

In some embodiments, one Z is

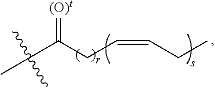

and r is 7.

In other embodiments, one Z is

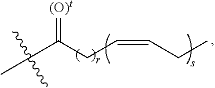

and s is 3.

In some embodiments, one Z is

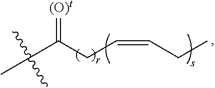

and s is 5.

In some embodiments, one Z is

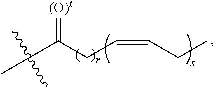

and s is 6.

In some embodiments, one Z is

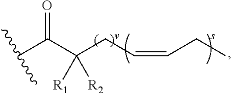

and v is 1.

In other embodiments, one Z is

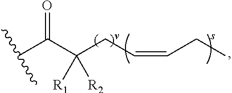

and v is 2.

In some embodiments, one Z is

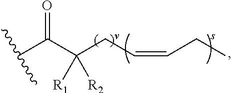

and v is 6.

In some embodiments, one Z is

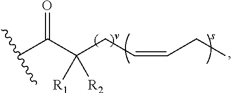

and s is 3.

In some embodiments, one Z is

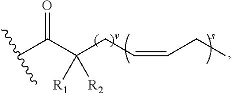

and s is 5.

In other embodiments, one Z is

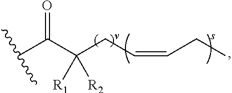

and s is 6.

In other embodiments, Z is

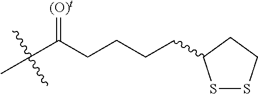

and t is 1.

In some embodiments, Z is

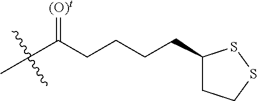

and t is 1.

In some embodiments, W₁ is NH.

In some embodiments, W₂ is NH.

In some embodiments, W₁ is O.

In some embodiments, W₂ is O.

In some embodiments, W₁ is null.

In some embodiments, W₂ is null.

In some embodiments, W₁ and W₂ are each NH.

In some embodiments, W₁ and W₂ are each null.

In some embodiments, W₁ is O and W₂ is NH.

In some embodiments, W₁ and W₂ are each NR, and R is CH₃.

In some embodiments, m is 0.

In other embodiments, m is 1.

In other embodiments, m is 2.

In some embodiments, L is —S— or —S—S—.

In some embodiments, L is —O—.

In some embodiments, L is —C(O)—.

In some embodiments, L is heteroaryl.

In some embodiments, L is heterocycle.

In some embodiments, L is

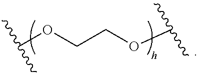

In some embodiments, L is

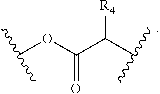

In some embodiments, L is

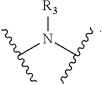

In some embodiments, L is

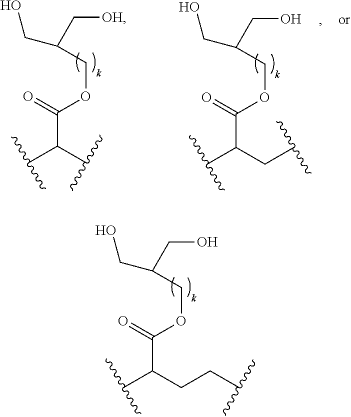

In some embodiments, L is

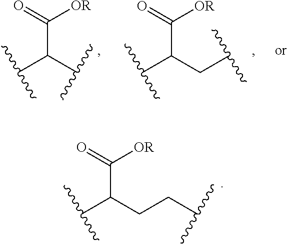

In some embodiments, L is

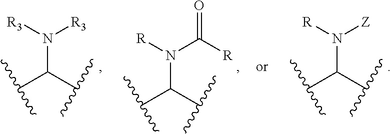

In some embodiments, L is

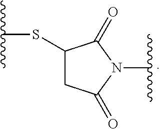 [SMILES: *SC1CC(=O)N(*)C1=O]

In some embodiments, L is

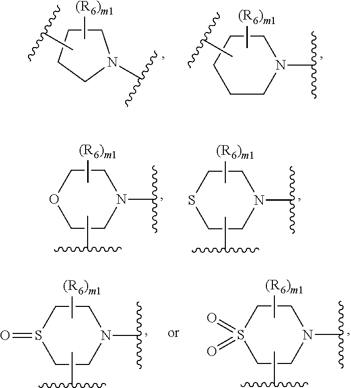

In some embodiments, L is

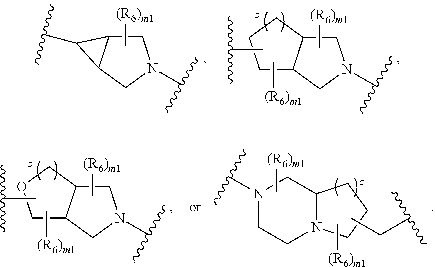

In some embodiments, L is

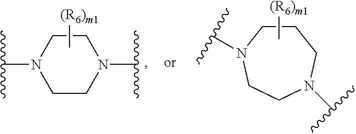

In some embodiments, L is

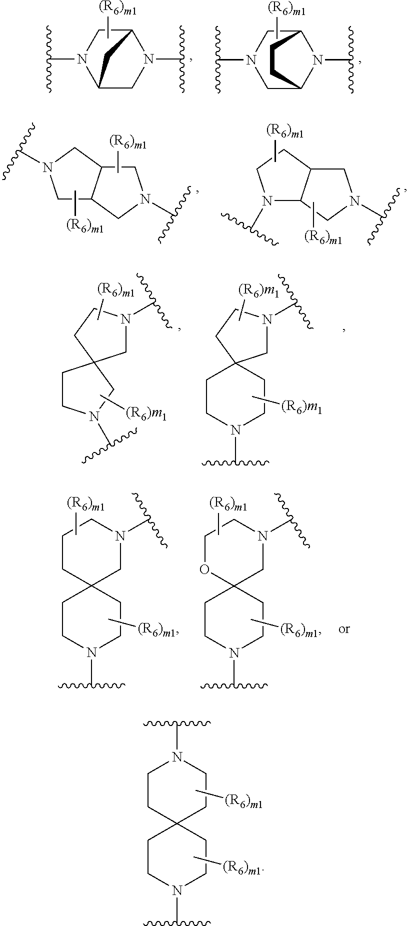

In other embodiments, one of n, o, p, and q is 1.

In some embodiments, two of n, o, p, and q are each 1.

In other embodiments, three of n, o, p, and q are each 1.

In some embodiments n, o, p, and q are each 1.

In some embodiments, one d is C(O)OR.

In some embodiments, r is 2 and s is 6.

In some embodiments, r is 3 and s is 5.

In some embodiments, t is 1.

In some embodiments, W₁ and W₂ are each NH, m is 0, n, and o are each 1, and p and q are each 0.

In some embodiments, W₁ and W₂ are each NH, m is 1, n, o, p, and q are each 1, and L is 0.

In some embodiments, W₁ and W₂ are each NH, m is 1, n, o, p, and q are each 1, and L is

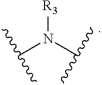

In some embodiments, W₁ and W₂ are each NH, m is 1, n, o, p, and q are each 1, and L is —S—S—.

In some embodiments, W₁ and W₂ are each NH, m is 1, n and o are each 0, p and q are each 1, and L is

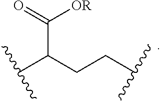

In some embodiments, W₁ and W₂ are each NH, m is 1, k is 0, n and o are each 0, p and q are each 1, and L is

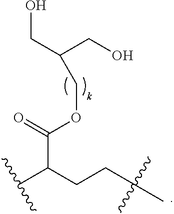

In some embodiments, W₁ and W₂ are each NH, m is 1, n and o are each 1, p and q are each 0, and L is

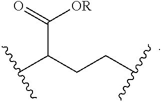

In some embodiments, W₁ and W₂ are each NH, m is 1, k is 0, n is 1, o, p and q are each 0, and L is

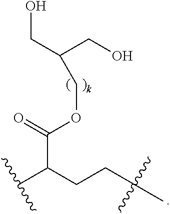

In some embodiments, W₁ and W₂ are each NH, m is 1, n, o, and p are each 0, and q is 1, and L is

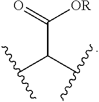

In some embodiments, W₁ and W₂ are each NH, m is 1, k is 1, n, o, and p are each 0, and q is 1, and L is

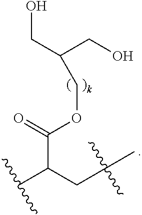

In some embodiments, W₁ and W₂ are each NH, m is 1, n is 1, and o, p, and q are each 0, and L is

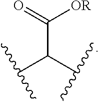

In some embodiments, W₁ and W₂ are each NH, m is 1, k is 1, o, p, and q are each 0, and L is

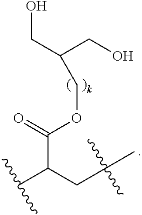

In some embodiments, W₁ and W₂ are each NH, m is 1, n, o, p, and q are each 1, and L is

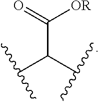

In some embodiments, W₁ and W₂ are each NH, m is 1, n, o, p, and q are each 1, and L is

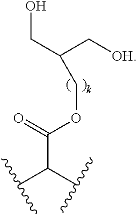

In some embodiments, W₁ and W₂ are each NH, m is 0, k is 1, o and p are each 1, and q is 0.

In some embodiments, W₁ and W₂ are each NH, m is 0, n, o, p, and q are each 1.

In some embodiments, W₁ and W₂ are each NH, m is 0, n and o are each 1, p and q are each 0, and each a is CH₃.

In some embodiments, W₁ and W₂ are each NH, m is 0, n and o are each 1, p and q are each 0, and each b is CH₃.

In some embodiments, W₁ and W₂ are each NH, m is 1, n, o, p, and q are each 1, R₃ is H, and L is

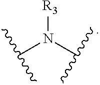

In some embodiments, W₁ and W₂ are each NH, m is 1, n, p and q are each 1, and o is 2, R₃ is H, and L is

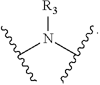

In some embodiments, W₁ and W₂ are each NH, m is 1, n, o, p are each 1, and q is 2, and L is

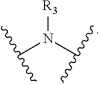

In some embodiments, W₁ and W₂ are each NH, m is 1, n, o, p, and q are each 1, and L is

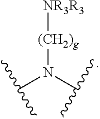

In some embodiments, W₁ and W₂ are each NH, m is 1, n and p are each 1, and o and q are each 0, and L is —C(O)—.

In some embodiments, W₁ and W₂ are each NH, m is 1, n and p are each 1, and o, and q are each 0, and L is

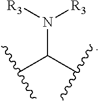

In some embodiments, W₁ and W₂ are each NH, m is 1, n, o, p, q are each 1, and L is

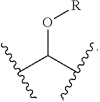

In some embodiments, W₁ and W₂ are each NH, m is 1, n, o, p, and q are each 1, h is 1, and L is

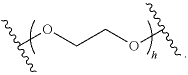

In some embodiments, W₁ and W₂ are each NH, m is 1, n, o, p, and q are each 1, and L is —S—.

In some embodiments, W₁ and W₂ are each NH, m is 1, n, o, p are each 0, q is 1, one d is —CH₃, and L is

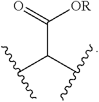

In some embodiments, W₁ and W₂ are each NH, m is 2, n, o, p, and q are each 0, one L is

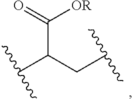

and
one L is

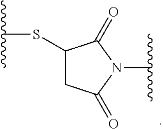 [SMILES: *SC1CC(=O)N(*)C1=O]

In some embodiments, m is 0, n, o, p, and q are each 0, and W₁ and W₂ are taken together to form an optionally substituted piperazine group.

In some embodiments, m is 1, n, o, p, and q are each 0, W₁ and W₂ are each null, and L is

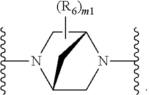

In some embodiments, m is 1, n and p are each 1, o and q are each 0, W₁ and W₂ are each NH, and L is C₃-C₆ cycloalkyl.

In some embodiments, m is 1, n is 1, o, p, and q are each 0, W₁ and W₂ are each NH, and L is C₃-C₆ cycloalkyl.

In some embodiments, m is 1, n, o, p, are each 0, q is 1, W₁ and W₂ are each NH, and L is C₃-C₆ cycloalkyl.

In some embodiments, m is 1, n, o, p, and q are each 0, W₁ is NH, W₂ is null, and L is

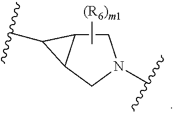

In some embodiments, m is 1, n o, p, and q are each 0, W₁ is null, W₂ is NH, and L is

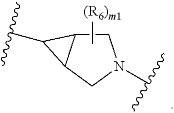

In some embodiments, m is 1, n o, p, and q are each 0, W₁ is NH, W₂ is null, and L is

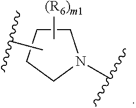

In some embodiments, m is 1, n o, p, and q are each 0, W₁ is null, W₂ is NH, and L is

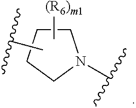

In some embodiments, m is 1, n is 1, o, p, and q are each 0, W₁ is NH, W₂ is null, and L is

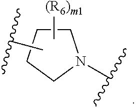

In some embodiments, m is 1, n, o, p, are each 0, q is 1, W₁ is null, W₂ is NH, and L is

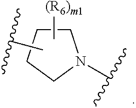

In some embodiments, m is 1, n, o, p, and q are each 0, W₁ is NH, W₂ is null, and L is

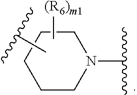

In some embodiments, m is 1, n, o, p, and q are each 0, W₁ is null, W₂ is NH, and L is

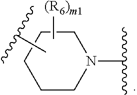

In some embodiments, m is 1, n is 1, o, p, and q are each 0, W₁ is NH, W₂ is null, and L is

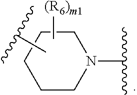

In some embodiments, m is 1, n, o, p, are each 0, q is 1, W₁ is null, W₂ is NH, and L is

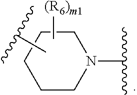

In some embodiments, m is 1, n is 1, o, p, and q are each 0, W₁ is NH, W₂ is null, and L is

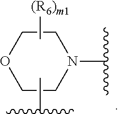

In some embodiments, m is 1, n, o, p, are each 0, q is 1, W₁ is null, W₂ is NH, and L is

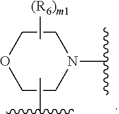

In some embodiments, m is 1, n, o, p, q are each 0, W₁ and W₂ is null, and L is

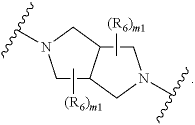

In some embodiments, m is 1, n, o, p, q are each 0, W₁ and W₂ is null, and L is

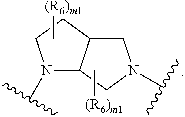

In some embodiments, m is 1, n, o, p, q are each 0, W₁ is NH, W₂ is null, and L is

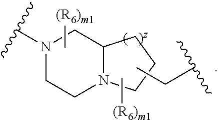

In some embodiments, m is 1, n, o, p, q are each 0, W₁ is null, W₂ is NH, and L is

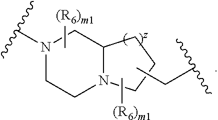

In some embodiments, m is 1, n, o, p, are each 0, q is 1, W₁ and W₂ are each and NH, is null, L is

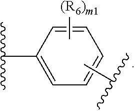

In some embodiments, m is 1, n, o, p, are each 0, q is 1, W₁ and W₂ are each NH, is null, and L is a heteroaryl.

In some of the foregoing embodiments, r is 2, s is 6 and t is 1.

In some of the foregoing embodiments, r is 3, s is 5 and t is 1.

In some of the foregoing embodiments, Z is

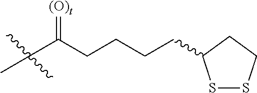

and

t is 1.

In Formula I and I′, any one or more of H may be substituted with a deuterium. It is also understood in Formula I and I′ that a methyl substituent can be substituted with a C₁-C₆ alkyl.

In other illustrative embodiments, compounds of Formula I are as set forth below:
(4Z,7Z,10Z,13Z,16Z,19Z)-docosa-4,7,10,13,16,19-hexaenoic acid [2-((4Z,7Z,10Z,13Z,16Z,19Z)-docosa-4,7,10,13,16,19-hexaenoylamino)-ethyl]-amide (I-1);
(4Z,7Z,10Z,13Z,16Z,19Z)-docosa-4,7,10,13,16,19-hexaenoic acid (2-{[2-((4Z,7Z,10Z,13Z,16Z,19Z)-docosa-4,7,10,13,16,19-hexaenoylamino)-ethyl]-methyl-amino}-ethyl)-amide (I-2);
(4Z,7Z,10Z,13Z,16Z,19Z)-Docosa-4,7,10,13,16,19-hexaenoic acid {2-[2-((4Z,7Z,10Z,13Z,16Z,19Z)-docosa-4,7,10,13,16,19-hexaenoylamino)-ethyldisulfanyl]-ethyl}-amide (I-3);
(S)-2,6-Bis-((4Z,7Z,10Z,13Z,16Z,19Z)-docosa-4,7,10,13,16,19-hexaenoylamino)-hexanoic acid methyl ester (I-4);
(S)-2,6-bis-((4Z,7Z,10Z,13Z,16Z,19Z)-docosa-4,7,10,13,16,19-hexaenoylamino)-hexanoic acid 2-hydroxy-1-hydroxymethyl-ethyl ester (I-5);
(S)-2,5-bis-((4Z,7Z,10Z,13Z,16Z,19Z)-docosa-4,7,10,13,16,19-hexaenoylamino)-pentanoic acid methyl ester (I-6);
(S)-2,5-bis-((4Z,7Z,10Z,13Z,16Z,19Z)-docosa-4,7,10,13,16,19-hexaenoylamino)-pentanoic acid (I-7);
(S)-2,5-bis-((4Z,7Z,10Z,13Z,16Z,19Z)-docosa-4,7,10,13,16,19-hexaenoylamino)-pentanoic acid 2-hydroxy-1-hydroxymethyl-ethyl ester (I-8);
(S)-2,3-bis-((4Z,7Z,10Z,13Z,16Z,19Z)-docosa-4,7,10,13,16,19-hexaenoylamino)-propionic acid methyl ester (I-9);
(S)-2,3-bis-((4Z,7Z,10Z,13Z,16Z,19Z)-docosa-4,7,10,13,16,19-hexaenoylamino)-propionic acid (I-10);
(S)-2,3-bis-((4Z,7Z,10Z,13Z,16Z,19Z)-docosa-4,7,10,13,16,19-hexaenoylamino)-propionic acid 2-hydroxy-1-hydroxymethyl-ethyl ester (I-11);
4-((4Z,7Z,10Z,13Z,16Z,19Z)-docosa-4,7,10,13,16,19-hexaenoylamino)-2-[2-((4Z,7Z,10Z,13Z,16Z,19Z)-docosa-4,7,10,13,16,19-hexaenoylamino)-ethyl]-butyric acid (I-12);
4-((4Z,7Z,10Z,13Z,16Z,19Z)-docosa-4,7,10,13,16,19-hexaenoylamino)-2-[2((4Z,7Z,10Z,13Z,16Z,19Z)-docosa-4,7,10,13,16,19-hexaenoylamino)-ethyl]-butyric acid 2-hydroxy-1-hydroxymethyl-ethyl ester (I-13);
(4Z,7Z,10Z,13Z,16Z,19Z)-docosa-4,7,10,13,16,19-hexaenoic acid {2-[[2-((4Z,7Z,10Z,13Z,16Z,19Z)-docosa-4,7,10,13,16,19-hexaenoylamino)-ethyl]-(2-morpholin-4-yl-ethyl)-amino]-ethyl}-amide (I-14);
(4Z,7Z,10Z,13Z,16Z,19Z)-docosa-4,7,10,13,16,19-hexaenoic acid {2-[[2-((4Z,7Z,10Z,13Z,16Z,19Z)-docosa-4,7,10,13,16,19-hexaenoylamino)-ethyl]-(3-piperazin-1-yl-propyl)-amino]-ethyl}-amide (I-15);
(4Z,7Z,10Z,13Z,16Z,19Z)-docosa-4,7,10,13,16,19-hexaenoic acid [3-((4Z,7Z,10Z,13Z,16Z,19Z)-docosa-4,7,10,13,16,19-hexaenoylamino)-2-oxo-propyl]-amide (I-16);
(4Z,7Z,10Z,13Z,16Z,19Z)-docosa-4,7,10,13,16,19-hexaenoic acid [3-((4Z,7Z,10Z,13Z,16Z,19Z)-docosa-4,7,10,13,16,19-hexaenoylamino)-2-morpholin-4-yl-propyl]-amide (I-17);
(4Z,7Z,10Z,13Z,16Z,19Z)-docosa-4,7,10,13,16,19-hexaenoic acid [3-((4Z,7Z,10Z,13Z,16Z,19Z)-docosa-4,7,10,13,16,19-hexaenoylamino)-2-piperazin-1-yl-propyl]-amide (I-18);
(4Z,7Z,10Z,13Z,16Z,19Z)-docosa-4,7,10,13,16,19-hexaenoic acid [5-((4Z,7Z,10Z,13Z,16Z,19Z)-docosa-4,7,10,13,16,19-hexaenoylamino)-3-hydroxy-pentyl]-amide (I-19);
(4Z,7Z,10Z,13Z,16Z,19Z)-docosa-4,7,10,13,16,19-hexaenoic acid [5-((4Z,7Z,10Z,13Z,16Z,19Z)-docosa-4,7,10,13,16,19-hexaenoylamino)-3-morpholin-4-yl-pentyl]-amide (I-20);
(4Z,7Z,10Z,13Z,16Z,19Z)-docosa-4,7,10,13,16,19-hexaenoic acid (2-{2-[2-((4Z,7Z,10Z,13Z,16Z,19Z)-docosa-4,7,10,13,16,19-hexaenoylamino)-ethoxy]-ethoxy}-ethyl)-amide (I-21);
(4Z,7Z,10Z,13Z,16Z,19Z)-docosa-4,7,10,13,16,19-hexaenoic acid {2-[2-((4Z,7Z,10Z,13Z,16Z,19Z)-docosa-4,7,10,13,16,19-hexaenoylamino)-ethylsulfanyl]-ethyl}-amide (I-22);
(2S,3R)-2-((4Z,7Z,10Z,13Z,16Z,19Z)-docosa-4,7,10,13,16,19-hexaenoylamino)-3-[(S)-2-((4Z,7Z,10Z,13Z,16Z,19Z)-docosa-4,7,10,13,16,19-hexaenoylamino)-propionyloxy]-butyric acid methyl ester (I-23);
(R)-2-((4Z,7Z,10Z,13Z,16Z,19Z)-docosa-4,7,10,13,16,19-hexaenoylamino)-3-{1-[2-((4Z,7Z,10Z,13Z,16Z,19Z)-docosa-4,7,10,13,16,19-hexaenoylamino)-ethyl]-2,5-dioxo-pyrrolidin-3-ylsulfanyl}-propionic acid methyl ester (I-24);
(4Z,7Z,10Z,13Z,16Z,19Z)-1-[4-((4Z,7Z,10Z,13Z,16Z,19Z)-docosa-4,7,10,13,16,19-hexaenoyl)-piperazin-1-yl]-docosa-4,7,10,13,16,19-hexaen-1-one (I-25);
(4Z,7Z,10Z,13Z,16Z,19Z)-1-[(3S,5R)-4-((4Z,7Z,10Z,13Z,16Z,19Z)-docosa-4,7,10,13,16,19-hexaenoyl)-3,5-dimethyl-piperazin-1-yl]-docosa-4,7,10,13,16,19-hexaen-1-one (I-26);
(4Z,7Z,10Z,13Z,16Z,19Z)-1-[(1S,4S)-5-((4Z,7Z,10Z,13Z,16Z,19Z)-docosa-4,7,10,13,16,19-hexaenoyl)-2,5-diaza-bicyclo[2.2.1]hept-2-yl]-docosa-4,7,10,13,16,19-hexaen-1-one (I-27);
(4Z,7Z,10Z,13Z,16Z,19Z)-docosa-4,7,10,13,16,19-hexaenoic acid {2-[((4Z,7Z,10Z,13Z,16Z,19Z)-docosa-4,7,10,13,16,19-hexaenoylamino)-methyl]-cyclopropylmethyl}-amide(I-28);
(4Z,7Z,10Z,13Z,16Z,19Z)-docosa-4,7,10,13,16,19-hexaenoic acid {4-[((4Z,7Z,10Z,13Z,16Z,19Z)-docosa-4,7,10,13,16,19-hexaenoylamino)-methyl]-cyclohexyl}-amide(I-29);
(4Z,7Z,10Z,13Z,16Z,19Z)-docosa-4,7,10,13,16,19-hexaenoic acid [3-((4Z,7Z,10Z,13Z,16Z,19Z)-docosa-4,7,10,13,16,19-hexaenoyl)-3-aza-bicyclo[3.1.0]hex-6-yl]-amide (I-30);
(4Z,7Z,10Z,13Z,16Z,19Z)-docosa-4,7,10,13,16,19-hexaenoic acid [(S)-1-((4Z,7Z,10Z,13Z,16Z,19Z)-docosa-4,7,10,13,16,19-hexaenoyl)-pyrrolidin-3-yl]-amide (I-31);
(4Z,7Z,10Z,13Z,16Z,19Z)-docosa-4,7,10,13,16,19-hexaenoic acid [1-((4Z,7Z,10Z,13Z,16Z,19Z)-docosa-4,7,10,13,16,19-hexaenoyl)-pyrrolidin-2-ylmethyl]-amide (I-32);
(4Z,7Z,10Z,13Z,16Z,19Z)-docosa-4,7,10,13,16,19-hexaenoic acid [1-((4Z,7Z,10Z,13Z,16Z,19Z)-docosa-4,7,10,13,16,19-hexaenoyl)-piperidin-4-yl]-amide (I-33)

(4Z,7Z,10Z,13Z,16Z,19Z)-docosa-4,7,10,13,16,19-hexaenoic acid [1-((4Z,7Z,10Z,13Z,16Z,19Z)-docosa-4,7,10,13,16,19-hexaenoyl)-piperidin-4-ylmethyl]-amide (I-34);
(4Z,7Z,10Z,13Z,16Z,19Z)-docosa-4,7,10,13,16,19-hexaenoic acid [1-((4Z,7Z,10Z,13Z,16Z,19Z)-docosa-4,7,10,13,16,19-hexaenoyl)-piperidin-2-ylmethyl]-amide (I-35);
(4Z,7Z,10Z,13Z,16Z,19Z)-docosa-4,7,10,13,16,19-hexaenoic acid [4-((4Z,7Z,10Z,13Z,16Z,19Z)-docosa-4,7,10,13,16,19-hexaenoyl)-morpholin-3-ylmethyl]-amide (I-36);
(4Z,7Z,10Z,13Z,16Z,19Z)-docosa-4,7,10,13,16,19-hexaenoic acid [2-((5Z,8Z,11Z,14Z,17Z)-icosa-5,8,11,14,17-pentaenoylamino)-ethyl]-amide (I-37);
(4Z,7Z,10Z,13Z,16Z,19Z)-docosa-4,7,10,13,16,19-hexaenoic acid {2-[2-((5Z,8Z,11Z,14Z,17Z)-icosa-5,8,11,14,17-pentaenoylamino)-ethoxy]-ethyl}-amide (I-38);
(4Z,7Z,10Z,13Z,16Z,19Z)-docosa-4,7,10,13,16,19-hexaenoic acid (2-{[2((5Z,8Z,11Z,14Z,17Z)-icosa-5,8,11,14,17-pentaenoylamino)-ethyl]-methyl-amino}-ethyl)-amide (I-39);
(4Z,7Z,10Z,13Z,16Z,19Z)-docosa-4,7,10,13,16,19-hexaenoic acid {2-[2-((5Z,8Z,11Z,14Z,17Z)-icosa-5,8,11,14,17-pentaenoylamino)-ethylamino]-ethyl}-amide (I-40);
(4Z,7Z,10Z,13Z,16Z,19Z)-docosa-4,7,10,13,16,19-hexaenoic acid {2-[2-((5Z,8Z,11Z,14Z,17Z)-icosa-5,8,11,14,17-pentaenoylamino)-ethyldisulfanyl]-ethyl}-amide(I-41);
(S)-6-((4Z,7Z,10Z,13Z,16Z,19Z)-docosa-4,7,10,13,16,19-hexaenoylamino)-2-((5Z,8Z,11Z,14Z,17Z)-icosa-5,8,11,14,17-pentaenoylamino)-hexanoic acid methyl ester (I-42);
(S)-2-((4Z,7Z,10Z,13Z,16Z,19Z)-docosa-4,7,10,13,16,19-hexaenoylamino)-6-((5Z,8Z,11Z,14Z,17Z)-icosa-5,8,11,14,17-pentaenoylamino)-hexanoic acid methyl ester (I-43);
(S)-6-((4Z,7Z,10Z,13Z,16Z,19Z)-docosa-4,7,10,13,16,19-hexaenoylamino)-2-((5Z,8Z,11Z,14Z,17Z)-icosa-5,8,11,14,17-pentaenoylamino)-hexanoic acid (I-44);
(S)-2-((4Z,7Z,10Z,13Z,16Z,19Z)-docosa-4,7,10,13,16,19-hexaenoylamino)-6-((5Z,8Z,11Z,14Z,17Z)-icosa-5,8,11,14,17-pentaenoylamino)-hexanoic acid (I-45);
(S)-6-((4Z,7Z,10Z,13Z,16Z,19Z)-docosa-4,7,10,13,16,19-hexaenoylamino)-2-((5Z,8Z,11Z,14Z,17Z)-icosa-5,8,11,14,17-pentaenoylamino)-hexanoic acid 2-hydroxy-1-hydroxymethyl-ethyl ester (I-46);
(S)-2-((4Z,7Z,10Z,13Z,16Z,19Z)-docosa-4,7,10,13,16,19-hexaenoylamino)-6-((5Z,8Z,11Z,14Z,17Z)-icosa-5,8,11,14,17-pentaenoylamino)-hexanoic acid 2-hydroxy-1-hydroxymethyl-ethyl ester (I-47);
(S)-2-((4Z,7Z,10Z,13Z,16Z,19Z)-docosa-4,7,10,13,16,19-hexaenoylamino)-5-((5Z,8Z,11Z,14Z,17Z)-icosa-5,8,11,14,17-pentaenoylamino)-pentanoic acid methyl ester (I-48);
(S)-5-((4Z,7Z,10Z,13Z,16Z,19Z)-docosa-4,7,10,13,16,19-hexaenoylamino)-2-((5Z,8Z,11Z,14Z,17Z)-icosa-5,8,11,14,17-pentaenoylamino)-pentanoic acid methyl ester (I-49);
(S)-2-((4Z,7Z,10Z,13Z,16Z,19Z)-docosa-4,7,10,13,16,19-hexaenoylamino)-5-((5Z,8Z,11Z,14Z,17Z)-icosa-5,8,11,14,17-pentaenoylamino)-pentanoic acid (I-50);
(S)-5-((4Z,7Z,10Z,13Z,16Z,19Z)-docosa-4,7,10,13,16,19-hexaenoylamino)-2-((5Z,8Z,11Z,14Z,17Z)-icosa-5,8,11,14,17-pentaenoylamino)-pentanoic acid (I-51);
(S)-2-((4Z,7Z,10Z,13Z,16Z,19Z)-docosa-4,7,10,13,16,19-hexaenoylamino)-5-((5Z,8Z,11Z,14Z,17Z)-icosa-5,8,11,14,17-pentaenoylamino)-pentanoic acid 2-hydroxy-1-hydroxymethyl-ethyl ester(I-52);
(S)-5-((4Z,7Z,10Z,13Z,16Z,19Z)-docosa-4,7,10,13,16,19-hexaenoylamino)-2-((5Z,8Z,11Z,14Z,17Z)-icosa-5,8,11,14,17-pentaenoylamino)-pentanoic acid 2-hydroxy-1-hydroxymethyl-ethyl ester (I-53);
2-[2-((4Z,7Z,10Z,13Z,16Z,19Z)-docosa-4,7,10,13,16,19-hexaenoylamino)-ethyl]-4-((5Z,8Z,11Z,14Z,17Z)-icosa-5,8,11,14,17-pentaenoylamino)-butyric acid (I-54);
2-[2-((4Z,7Z,10Z,13Z,16Z,19Z)-docosa-4,7,10,13,16,19-hexaenoylamino)-ethyl]-4-((5Z,8Z,11Z,14Z,17Z)-icosa-5,8,11,14,17-pentaenoylamino)-butyric acid 2-hydroxy-1-hydroxymethyl-ethyl ester (I-55);
(4Z,7Z,10Z,13Z,16Z,19Z)-docosa-4,7,10,13,16,19-hexaenoic acid {2-[[2-((5Z,8Z,11Z,14Z,17Z)-icosa-5,8,11,14,17-pentaenoylamino)-ethyl]-(2-morpholin-4-yl-ethyl)-amino]-ethyl}-amide (I-56);
(4Z,7Z,10Z,13Z,16Z,19Z)-docosa-4,7,10,13,16,19-hexaenoic acid {2-[[2-((5Z,8Z,11Z,14Z,17Z)-icosa-5,8,11,14,17-pentaenoylamino)-ethyl]-(3-piperazin-1-yl-propyl)-amino]-ethyl}-amide (I-57);
(4Z,7Z,10Z,13Z,16Z,19Z)-docosa-4,7,10,13,16,19-hexaenoic acid [3-((5Z,8Z,11Z,14Z,17Z)-icosa-5,8,11,14,17-pentaenoylamino)-2-oxo-propyl]-amide (I-58);
(4Z,7Z,10Z,13Z,16Z,19Z)-docosa-4,7,10,13,16,19-hexaenoic acid [3-((5Z,8Z,11Z,14Z,17Z)-icosa-5,8,11,14,17-pentaenoylamino)-2-morpholin-4-yl-propyl]-amide (I-59);
(4Z,7Z,10Z,13Z,16Z,19Z)-docosa-4,7,10,13,16,19-hexaenoic acid [3-((5Z,8Z,11Z,14Z,17Z)-icosa-5,8,11,14,17-pentaenoylamino)-2-piperazin-1-yl-propyl]-amide (I-60);
(4Z,7Z,10Z,13Z,16Z,19Z)-docosa-4,7,10,13,16,19-hexaenoic acid [3-hydroxy-5-((5Z,8Z,11Z,14Z,17Z)-icosa-5,8,11,14,17-pentaenoylamino)-pentyl]-amide(I-61);
(4Z,7Z,10Z,13Z,16Z,19Z)-docosa-4,7,10,13,16,19-hexaenoic acid [5-((5Z,8Z,11Z,14Z,17Z)-icosa-5,8,11,14,17-pentaenoylamino)-3-morpholin-4-yl-pentyl]-amide (I-62);
(4Z,7Z,10Z,13Z,16Z,19Z)-docosa-4,7,10,13,16,19-hexaenoic acid (2-{2-[2-((5Z,8Z,11Z,14Z,17Z)-icosa-5,8,11,14,17-pentaenoylamino)-ethoxy]-ethoxy}-ethyl)-amide (I-63);
(4Z,7Z,10Z,13Z,16Z,19Z)-docosa-4,7,10,13,16,19-hexaenoic acid {2-[2-((5Z,8Z,11Z,14Z,17Z)-icosa-5,8,11,14,17-pentaenoylamino)-ethylsulfanyl]-ethyl}-amide (I-64);
(2S,3R)-methyl 2-((4Z,7Z,10Z,13Z,16Z,19Z)-docosa-4,7,10,13,16,19-hexaenamido)-3-((S)-2-((5Z,8Z,11Z,14Z,17Z)-icosa-5,8,11,14,17-pentaenamido)propanoyloxy)butanoate(I-65);
(2S,3R)-methyl 3-((S)-2-((4Z,7Z,10Z,13Z,16Z,19Z)-docosa-4,7,10,13,16,19-hexaenamido)propanoyloxy)-2-((5Z,8Z,11Z,14Z,17Z)-icosa-5,8,11,14,17-pentaenamido)butanoate (I-66);
(4Z,7Z,10Z,13Z,16Z,19Z)-1-[4-((5Z,8Z,11Z,14Z,17Z)-icosa-5,8,11,14,17-pentaenoyl)-piperazin-1-yl]-docosa-4,7,10,13,16,19-hexaen-1-one (I-67);
(4Z,7Z,10Z,13Z,16Z,19Z)-1-[(2S,6R)-4-((5Z,8Z,11Z,14Z,17Z)-icosa-5,8,11,14,17-pentaenoyl)-2,6-dimethyl-piperazin-1-yl]-docosa-4,7,10,13,16,19-hexaen-1-one (I-68);
(4Z,7Z,10Z,13Z,16Z,19Z)-1-[(3S,5R)-4-((5Z,8Z,11Z,14Z,17Z)-icosa-5,8,11,14,17-pentaenoyl)-3,5-dimethyl-piperazin-1-yl]-docosa-4,7,10,13,16,19-hexaen-1-one (I-69);
(4Z,7Z,10Z,13Z,16Z,19Z)-1-[(1S,4S)-5-((5Z,8Z,11Z,14Z,17Z)-icosa-5,8,11,14,17-pentaenoyl)-2,5-diaza-bicyclo[2.2.1]hept-2-yl]-docosa-4,7,10,13,16,19-hexaen-1-one (I-70);
(4Z,7Z,10Z,13Z,16Z,19Z)-docosa-4,7,10,13,16,19-hexaenoic acid [(S)-1-((5Z,8Z,11Z,14Z,17Z)-icosa-5,8,11,14,17-pentaenoyl)-pyrrolidin-3-yl]-amide (I-71);
(5Z,8Z,11Z,14Z,17Z)-icosa-5,8,11,14,17-pentaenoic acid [(S)-1-((4Z,7Z,10Z,13Z,16Z,19Z)-docosa-4,7,10,13,16,19-hexaenoyl)-pyrrolidin-3-yl]-amide (I-72);
(4Z,7Z,10Z,13Z,16Z,19Z)-docosa-4,7,10,13,16,19-hexaenoic acid [1-((5Z,8Z,11Z,14Z,17Z)-icosa-5,8,11,14,17-pentaenoyl)-pyrrolidin-2-ylmethyl]-amide (I-73);
(5Z,8Z,11Z,14Z,17Z)-icosa-5,8,11,14,17-pentaenoic acid [1-((4Z,7Z,10Z,13Z,16Z,19Z)-docosa-4,7,10,13,16,19-hexaenoyl)-pyrrolidin-2-ylmethyl]-amide (I-74);
(4Z,7Z,10Z,13Z,16Z,19Z)-docosa-4,7,10,13,16,19-hexaenoic acid [1-((5Z,8Z,11Z,14Z,17Z)-icosa-5,8,11,14,17-pentaenoyl)-piperidin-4-yl]-amide (I-75);
(5Z,8Z,11Z,14Z,17Z)-icosa-5,8,11,14,17-pentaenoic acid [1-((4Z,7Z,10Z,13Z,16Z,19Z)-docosa-4,7,10,13,16,19-hexaenoyl)-piperidin-4-yl]-amide (I-76);
(4Z,7Z,10Z,13Z,16Z,19Z)-docosa-4,7,10,13,16,19-hexaenoic acid [1-((5Z,8Z,11Z,14Z,17Z)-icosa-5,8,11,14,17-pentaenoyl)-piperidin-4-ylmethyl]-amide (I-77);
(5Z,8Z,11Z,14Z,17Z)-icosa-5,8,11,14,17-pentaenoic acid [1-((4Z,7Z,10Z,13Z,16Z,19Z)-docosa-4,7,10,13,16,19-hexaenoyl)-piperidin-4-ylmethyl]-amide (I-78);
(4Z,7Z,10Z,13Z,16Z,19Z)-docosa-4,7,10,13,16,19-hexaenoic acid [1-((5Z,8Z,11Z,14Z,17Z)-icosa-5,8,11,14,17-pentaenoyl)-piperidin-2-ylmethyl]-amide (I-79);
(5Z,8Z,11Z,14Z,17Z)-icosa-5,8,11,14,17-pentaenoic acid [1-((4Z,7Z,10Z,13Z,16Z,19Z)-docosa-4,7,10,13,16,19-hexaenoyl)-piperidin-2-ylmethyl]-amide (I-80);
(5Z,8Z,11Z,14Z,17Z)-icosa-5,8,11,14,17-pentaenoic acid [2-((5Z,8Z,11Z,14Z,17Z)-icosa-5,8,11,14,17-pentaenoylamino)-ethyl]-amide (I-81);
(5Z,8Z,11Z,14Z,17Z)-icosa-5,8,11,14,17-pentaenoic acid (2-{[2-((5Z,8Z,11Z,14Z,17Z)-icosa-5,8,11,14,17-pentaenoylamino)-ethyl]-methyl-amino}-ethyl)-amide (I-82);
(5Z,8Z,11Z,14Z,17Z)-icosa-5,8,11,14,17-pentaenoic acid {2-[2-((5Z,8Z,11Z,14Z,17Z)-icosa-5,8,11,14,17-pentaenoylamino)-ethyldisulfanyl]-ethyl}-amide (I-83);
(S)-2,6-bis-((5Z,8Z,11Z,14Z,17Z)-icosa-5,8,11,14,17-pentaenoylamino)-hexanoic acid methyl ester (I-84);
(S)-2,6-bis-((5Z,8Z,11Z,14Z,17Z)-icosa-5,8,11,14,17-pentaenoylamino)-hexanoic acid (I-85);
(S)-2,6-bis-((5Z,8Z,11Z,14Z,17Z)-icosa-5,8,11,14,17-pentaenoylamino)-hexanoic acid 2-hydroxy-1-hydroxymethyl-ethyl ester (I-86);
(S)-2,5-bis-((5Z,8Z,11Z,14Z,17Z)-icosa-5,8,11,14,17-pentaenoylamino)-pentanoic acid methyl ester (I-87);
(S)-2,5-bis-((5Z,8Z,11Z,14Z,17Z)-icosa-5,8,11,14,17-pentaenoylamino)-pentanoic acid (I-88);
(S)-2,5-bis-((5Z,8Z,11Z,14Z,17Z)-icosa-5,8,11,14,17-pentaenoylamino)-pentanoic acid ((I-89);
(8Z,11Z,14Z,17Z,20Z)-(2S,3S)-3-[(S)-2-((5Z,8Z,11Z,14Z,17Z)-icosa-5,8,11,14,17-pentaenoylamino)-propionyloxy]-2-methylamino-4-oxo-tricosa-8,11,14,17,20-pentaenoic acid methyl ester (I-90);
(5Z,8Z,11Z,14Z,17Z)-1-[4-((5Z,8Z,11Z,14Z,17Z)-icosa-5,8,11,14,17-pentaenoyl)-piperazin-1-yl]-icosa-5,8,11,14,17-pentaen-1-one (I-91);
(5Z,8Z,11Z,14Z,17Z)-icosa-5,8,11,14,17-pentaenoic acid [(S)-1-((5Z,8Z,11Z,14Z,17Z)-icosa-5,8,11,14,17-pentaenoyl)-pyrrolidin-3-yl]-amide (I-92);
(5Z,8Z,11Z,14Z,17Z)-icosa-5,8,11,14,17-pentaenoic acid [1-((5Z,8Z,11Z,14Z,17Z)-icosa-5,8,11,14,17-pentaenoyl)-pyrrolidin-2-ylmethyl]-amide (I-93);
(5Z,8Z,11Z,14Z,17Z)-icosa-5,8,11,14,17-pentaenoic acid [1-((5Z,8Z,11Z,14Z,17Z)-icosa-5,8,11,14,17-pentaenoyl)-piperidin-4-yl]-amide (I-94);
(5Z,8Z,11Z,14Z,17Z)-icosa-5,8,11,14,17-pentaenoic acid [1-((5Z,8Z,11Z,14Z,17Z)-icosa-5,8,11,14,17-pentaenoyl)-piperidin-4-ylmethyl]-amide (I-95);
(5Z,8Z,11Z,14Z,17Z)-icosa-5,8,11,14,17-pentaenoic acid [1-((5Z,8Z,11Z,14Z,17Z)-icosa-5,8,11,14,17-pentaenoyl)-piperidin-2-ylmethyl]-amide (I-96);
(5Z,8Z,11Z,14Z,17Z)-icosa-5,8,11,14,17-pentaenoic acid [2-((R)-5-[1,2]dithiolan-3-yl-pentanoylamino)-ethyl]-amide (I-97);
(4Z,7Z,10Z,13Z,16Z,19Z)-docosa-4,7,10,13,16,19-hexaenoic acid [2-((R)-5-[1,2]dithiolan-3-yl-pentanoylamino)-ethyl]-amide (I-98);
(5Z,8Z,11Z,14Z,17Z)-icosa-5,8,11,14,17-pentaenoic acid {2-[2-((R)-5-[1,2]dithiolan-3-yl-pentanoylamino)-ethoxy]-ethyl}-amide (I-99);
(4Z,7Z,10Z,13Z,16Z,19Z)-docosa-4,7,10,13,16,19-hexaenoic acid {2-[2-((R)-5-[1,2]dithiolan-3-yl-pentanoylamino)-ethoxy]-ethyl}-amide (I-100);
(5Z,8Z,11Z,14Z,17Z)-icosa-5,8,11,14,17-pentaenoic acid {2-[2-((R)-5-[1,2]dithiolan-3-yl-pentanoylamino)-ethylamino]-ethyl}-amide (I-101);
(4Z,7Z,10Z,13Z,16Z,19Z)-docosa-4,7,10,13,16,19-hexaenoic acid {2-[2-((R)-5-[1,2]dithiolan-3-yl-pentanoylamino)-ethylamino]-ethyl}-amide (I-102);
(5Z,8Z,11Z,14Z,17Z)-icosa-5,8,11,14,17-pentaenoic acid (2-{[2-((R)-5-[1,2]dithiolan-3-yl-pentanoylamino)-ethyl]-methyl-amino}-ethyl)-amide (I-103);
(4Z,7Z,10Z,13Z,16Z,19Z)-docosa-4,7,10,13,16,19-hexaenoic acid (2-{[2-((R)-5-[1,2]dithiolan-3-yl-pentanoylamino)-ethyl]-methyl-amino}-ethyl)-amide (I-104);
(5Z,8Z,11Z,14Z,17Z)-icosa-5,8,11,14,17-pentaenoic acid {2-[2-((R)-5-[1,2]dithiolan-3-yl-pentanoylamino)-ethyldisulfanyl]-ethyl}-amide (I-105);
(4Z,7Z,10Z,13Z,16Z,19Z)-docosa-4,7,10,13,16,19-hexaenoic acid {2-[2-((R)-5-[1,2] dithiolan-3-yl-pentanoylamino)-ethyldisulfanyl]-ethyl}-amide (I-106);
(S)-6-((R)-5-[1,2]dithiolan-3-yl-pentanoylamino)-2-((5Z,8Z,11Z,14Z,17Z)-icosa-5,8,11,14,17-pentaenoylamino)-hexanoic acid (I-107);
(S)-2-((R)-5-[1,2]dithiolan-3-yl-pentanoylamino)-6-((5Z,8Z,11Z,14Z,17Z)-icosa-5,8,11,14,17-pentaenoylamino)-hexanoic acid (I-108);
(S)-6-((R)-5-[1,2]dithiolan-3-yl-pentanoylamino)-2-((4Z,7Z,10Z,13Z,16Z,19Z)-docosa-4,7,10,13,16,19-hexaenoylamino)-hexanoic acid (I-109);
(S)-2-((R)-5-[1,2]dithiolan-3-yl-pentanoylamino)-6-((4Z,7Z,10Z,13Z,16Z,19Z)-docosa-4,7,10,13,16,19-hexaenoylamino)-hexanoic acid (I-110);
(S)-6-((R)-5-[1,2]dithiolan-3-yl-pentanoylamino)-2-((5Z,8Z,11Z,14Z,17Z)-icosa-5,8,11,14,17-pentaenoylamino)-hexanoic acid 2-hydroxy-1-hydroxymethyl-ethyl ester (I-111);
(S)-2-((R)-5-[1,2]dithiolan-3-yl-pentanoylamino)-6-((5Z,8Z,11Z,14Z,17Z)-icosa-5,8,11,14,17-pentaenoylamino)-hexanoic acid 2-hydroxy-1-hydroxymethyl-ethyl ester (I-112);
(S)-6-((R)-5-[1,2]dithiolan-3-yl-pentanoylamino)-2-((4Z,7Z,10Z,13Z,16Z,19Z)-docosa-4,7,10,13,16,19-hexaenoylamino)-hexanoic acid 2-hydroxy-1-hydroxymethyl-ethyl ester(I-113);
(S)-2-((R)-5-[1,2]dithiolan-3-yl-pentanoylamino)-6-((4Z,7Z,10Z,13Z,16Z,19Z)-docosa-4,7,10,13,16,19-hexaenoylamino)-hexanoic acid 2-hydroxy-1-hydroxymethyl-ethyl ester (I-114);
(S)-5-((R)-5-[1,2]dithiolan-3-yl-pentanoylamino)-2-((4Z,7Z,10Z,13Z,16Z,19Z)-docosa-4,7,10,13,16,19-hexaenoylamino)-pentanoic acid (I-115);
(S)-2-((R)-5-[1,2]dithiolan-3-yl-pentanoylamino)-5-((4Z,7Z,10Z,13Z,16Z,19Z)-docosa-4,7,10,13,16,19-hexaenoylamino)-pentanoic acid (I-116);
(S)-5-((R)-5-[1,2]dithiolan-3-yl-pentanoylamino)-2-((5Z,8Z,11Z,14Z,17Z)-icosa-5,8,11,14,17-pentaenoylamino)-pentanoic acid (I-117);
(S)-2-((R)-5-[1,2]dithiolan-3-yl-pentanoylamino)-5-((5Z,8Z,11Z,14Z,17Z)-icosa-5,8,11,14,17-pentaenoylamino)-pentanoic acid (I-118);
(S)-5-(R)-5-[1,2]dithiolan-3-yl-pentanoylamino)-2-((4Z,7Z,10Z,13Z,16Z,19Z)-docosa-4,7,10,13,16,19-hexaenoylamino)-pentanoic acid 2-hydroxy-1-hydroxymethyl-ethyl ester (I-119);
(S)-2-(R)-5-[1,2]dithiolan-3-yl-pentanoylamino)-5-((4Z,7Z,10Z,13Z,16Z,19Z)-docosa-4,7,10,13,16,19-hexaenoylamino)-pentanoic acid 2-hydroxy-1-hydroxymethyl-ethyl ester (I-120);
(S)-5-((R)-5-[1,2]dithiolan-3-yl-pentanoylamino)-2-((5Z,8Z,11Z,14Z,17Z)-icosa-5,8,11,14,17-pentaenoylamino)-pentanoic acid 2-hydroxy-1-hydroxymethyl-ethyl ester (I-121); and
(S)-2-(R)-5-[1,2]dithiolan-3-yl-pentanoylamino)-545Z,8Z,11Z,14Z,17Z)-icosa-5,8,11,14,17-pentaenoylamino)-pentanoic acid 2-hydroxy-1-hydroxymethyl-ethyl ester (I-122).

### Methods for Using Bis Fattyacid Conjugates

Also provided in the invention is a method for inhibiting, preventing, or treating inflammation or an inflammatory disease in a subject. The inflammation can be associated with an inflammatory disease or a disease where inflammation contributes to the disease. Inflammatory diseases can arise where there is an inflammation of the body tissue. These include local inflammatory responses and systemic inflammation. Examples of such diseases include, but are not limited to: organ transplant rejection; reoxygenation injury resulting from organ transplantation (see Grupp et al., J. Mol. Cell. Cardiol. 31: 297-303 (1999)) including, but not limited to, transplantation of the following organs: heart, lung, liver and kidney; chronic inflammatory diseases of the joints, including arthritis, rheumatoid arthritis, osteoarthritis and bone diseases associated with increased bone resorption; inflammatory bowel diseases such as ileitis, ulcerative colitis, Barrett's syndrome, and Crohn's disease; inflammatory lung diseases such as asthma, adult respiratory distress syndrome, chronic obstructive airway disease, and cystic fibrosis; inflammatory diseases of the eye including corneal dystrophy, trachoma, onchocerciasis, uveitis, sympathetic ophthalmitis and endophthalmitis; chronic inflammatory diseases of the gum, including gingivitis and periodontitis; chronic kidney disease (CKD); IgA nephropathy; inflammatory diseases of the kidney including uremic complications, glomerulonephritis and nephrosis; inflammatory diseases of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, amyotrophic lateral sclerosis and viral or autoimmune encephalitis. Metabolic disease such as type II diabetes mellitus; the prevention of type I diabetes; dyslipedemia; hypertriglyceridemia; diabetic complications, including, but not limited to glaucoma, retinopathy, macula edema, nephropathy, such as microalbuminuria and progressive diabetic nephropathy, polyneuropathy, diabetic neuropathy, atherosclerotic coronary arterial disease, peripheral arterial disease, nonketotic hyperglycemichyperosmolar coma, mononeuropathies, autonomic neuropathy, joint problems, and a skin or mucous membrane complication, such as an infection, a shin spot, a candidal infection or necrobiosis lipoidica diabeticorum; immune-complex vasculitis, systemic lupus erythematosus; inflammatory diseases of the heart such as cardiomyopathy, ischemic heart disease hypercholesterolemia, and atherosclerosis; as well as various other diseases that can have significant inflammatory components, including preeclampsia; chronic liver failure, brain and spinal cord trauma, and cancer. The inflammatory disease can also be a systemic inflammation of the body, exemplified by gram-positive or gram negative shock, hemorrhagic or anaphylactic shock, or shock induced by cancer chemotherapy in response to proinflammatory cytokines, e.g., shock associated with proinflammatory cytokines. Such shock can be induced, e.g., by a chemotherapeutic agent that is administered as a treatment for cancer. Other disorders include depression, obesity, allergic diseases, acute cardiovascular events, arrhythmia, prevention of sudden death.

In some embodiments, other diseases susceptible to treatment with bis-Fatty Acid Derivative are muscle wasting diseases such as Muscular Dystrophy including but not limited to Duchenne's Muscular Dystrophy, Becker Muscular Dystrophy, Emery-Dreifuss Muscular Dystrophy, Limb-Girdle Muscular Dystrophy, Facioscapulohumeral Muscular Dystrophy, Myotonic Dystrophy, Oculopharyngeal Muscular Dystrophy, Distal Muscular Dystrophy, Congential Muscular Dystrophy, Spinal Muscular Atrophy, and Spinal Bulbar Muscular Dystrophy. Other diseases that can be treated with bis-Fatty Acid Derivative include inflammatory myopathies such as dermatomositis, inclusion body myositis, and polymyositis, and cancer cachexia. Also inflammation that results from surgery and trauma can be treated with a bis-Fatty Acid Derivative. The compounds described herein are also useful in treating a variety of cancer such as carcinoma, sarcoma, lymphoma, leukemia, melanoma, mesothelioma, multiople myeloma, seminoma, and cancer of the bladder, blood, bone, brain, breast, central nervous system, colon, endometrium, esophagus, genitourinary tract, head, larynx, liver, lung, neck, ovary, pancreas, prostate, testicle, spleen, small intestine, large intestine or stomach. Still other diseases that can be treated with bis-fatty acid conjugates include fatty liver disease, non-alcoholic fatty liver disease, NASH (non-alcoholic steatohepatitis), Sarcopenia, Sjogren syndrome, (Chronic kidney disease has already mentioned above), Myasthenia gravis, and xerophthalmia.

In some embodiments, the subject is administered an effective amount of a bis-fatty acid conjugate.

The invention also includes pharmaceutical compositions useful for treating or preventing a metabolic disease, or for inhibiting a metabolic disease, or more than one of these activities. The compositions can be suitable for internal use and comprise an effective amount of a bis-fatty acid conjugate of Formula I′ and a pharmaceutically acceptable carrier. The bis-fatty acid conjugates are especially useful in that they demonstrate very low peripheral toxicity or no peripheral toxicity.

Compositions can be prepared according to conventional mixing, granulating or coating methods, respectively, and the present pharmaceutical compositions can contain from about 0.1% to about 90%, from about 10% to about 90%, or from about 30% to about 90% of the bis-fatty acid conjugate by weight or volume.

The bis-fatty acid conjugates can each be administered in amounts that are sufficient to treat or prevent a metabolic disease or prevent the development thereof in subjects.

Administration of the bis-fatty acid conjugates can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Illustrative pharmaceutical compositions are tablets and gelatin capsules comprising a bis-fatty acid conjugate and a pharmaceutically acceptable carrier, such as: a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl—cyclodextrin, PEG400, PEG200.

Liquid, particularly injectable, compositions can, for example, be prepared by dissolution, dispersion, etc. For example, the bis-fatty acid conjugate is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the bis-fatty acid conjugates.

The bis-fatty acid conjugates can be also formulated as a suppository that can be prepared from fatty emulsions or suspensions; using polyalkylene glycols such as propylene glycol, as the carrier.

The bis-fatty acid conjugates can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564, the contents of which are hereby incorporated in their entirety.

Bis-fatty acid conjugates can also be delivered by the use of monoclonal antibodies as individual carriers to which the bis-fatty acid conjugates are coupled. The bis-fatty acid conjugates can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the bis-fatty acid conjugates can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, bis-fatty acid conjugates are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

The dosage regimen utilizing the bis-fatty acid conjugate is selected in accordance with a variety of factors including type, species, age, weight, sex and medical condition of the patient; the severity of the condition to be treated; the route of administration; the renal or hepatic function of the patient; and the particular bis-fatty acid conjugate employed. A physician or veterinarian of ordinary skill in the art can readily determine and prescribe the effective amount of the drug required to prevent, counter or arrest the progress of the condition.

Effective dosage amounts of the present invention, when used for the indicated effects, range from about 20 mg to about 5,000 mg of the bis-fatty acid conjugate per day. Compositions for in vivo or in vitro use can contain about 20, 50, 75, 100, 150, 250, 500, 750, 1,000, 1,250, 2,500, 3,500, or 5,000 mg of the bis-fatty acid conjugate. In one embodiment, the compositions are in the form of a tablet that can be scored. Effective plasma levels of the bis-fatty acid derivative can range from about 5 ng/mL to 5000 ng/mL. Appropriate dosages of the bis-fatty acid conjugates can be determined as set forth in Goodman, L. S.; Gilman, A. The Pharmacological Basis of Therapeutics, 5th ed.; MacMillan: New York, 1975, pp. 201-226.

Bis-fatty acid conjugates can be administered in a single daily dose, or the total daily dosage can be administered in divided doses of two, three or four times daily. Furthermore, bis-fatty acid conjugates can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of the bis-fatty acid conjugate ranges from about 0.1% to about 15%, w/w or w/v.

### Combination Therapies

Bis-fatty acid conjugates may also be administered with other therapeutic agents such as cholesterol-lowering agents, fibrates and hypolipidemic agents, DPP-IV inhibitors as anti-diabetic agents, anti-diabetic agents, antiepileptic agents, antiglaucoma agents, antihypertensive agents, anti-inflammatory agents, TNF-α inhibitors, anti-depressant agents, anti-cancer agents, immunosuppressant agents, agents to treat osteoporosis, and agents to treat multiple sclerosis. In some embodiments, the bis-fatty acid conjuagte can be co-administered with the other therapeutic agent. In some embodiments, the bis-fatty acid conjugate can be administered before the other therapeutic agent. In some embodiments, the bis-fatty acid conjugate can be administered after the other therapeutic agent.

In some embodiments, the other therapeutic agent is a cholesterol-lowering agent. Non limiting examples of cholesterol-lowering agents are atorvastatin, cerivastatin, fluvastatin, lovastatin, pitavastatin, pravastatin, rosuvastatin, simvastatin, ezetimibe, and the combination of ezetimibe/simvastatin (Vytorin®).

In some embodiments, the other therapeutic agent is a fibrate or hypolipidemic agent. Non-limiting examples of fibrates or hypolipidemic agents are acifran, acipimox, beclobrate, bezafibrate, binifibrate, ciprofibrate, clofibrate, colesevelam, gemfibrozil, fenofibrate, melinamide, niacin, and ronafibrate.

In some embodiments, the other therapeutic agent is a DPP-IV inhibitor as anti-diabetic agent. Non-limiting examples of DPP-IV inhibitors as anti-diabetic agents are sitagliptin, saxagliptin, vildagliptin, linagliptin, dutogliptin, gemigliptin and alogliptin.

In some embodiments, the other therapeutic agent is an anti-diabetic agent. Non-limiting examples of anti-diabetic agents are acarbose, epalrestat, exenatide, glimepiride, liraglutide, metformin, miglitol, mitiglinide, nateglinide, pioglitazone, pramlintide, repaglinide, rosiglitazone, tolrestat, troglitazone, and voglibose.

In some embodiments, the other therapeutic agent is an antiepileptic agent. Non-limiting examples of antiepileptic agents include Gabapentin, pregabalin.

In some embodiments, the other therapeutic agent is an antiglaucoma agent. Non-limiting examples of antiglaucoma agents include apraclonidine, befunolol, bimatroprost, brimonidine, brinzolamide, dapiprazole, dorzolamide, latanoprost, levobunolol, tafluprost, travoprost, and unoprostone isopropyl ester.

In some embodiments, the other therapeutic agent is an antihypertensive agent. Non-limiting examples of antihypertensive agents include alacepril, alfuzosin, aliskiren, amlodipine besylate, amosulalol, aranidipine, arotinolol HCl, azelnidipine, barnidipine hydrochloride, benazepril hydrochloride, benidipine hydrochloride, betaxolol HCl, bevantolol HCl, bisoprolol fumarate, bopindolol, bosentan, budralazine, bunazosin HCl, candesartan cilexetil, captopril, carvedilol, celiprolol HCl, cicletanine, cilazapril, cinildipine, clevidipine, delapril, dilevalol, doxazosin mesylate, efonidipine, enalapril maleate, enalaprilat, eplerenone, eprosartan, felodipine, fenoldopam mesylate, fosinopril sodium, guanadrel sulfate, imidapril HCl, irbesartan, isradipine, ketanserin, lacidipine, lercanidipine, lisinopril, losartan, manidipine hydrochloride, mebefradil hydrochloride, moxonidine, nebivolol, nilvadipine, nipradilol, nisoldipine, olmesartan medoxomil, perindopril, pinacidil, quinapril, ramipril, rilmedidine, spirapril HCl, telmisartan, temocarpil, terazosin HCl, tertatolol HCl, tiamenidine HCl, tilisolol hydrochloride, trandolapril, treprostinil sodium, trimazosin HCl, valsartan, and zofenopril calcium.

In some embodiments, the other therapeutic agent is an anti-inflammatory agent. Non-limiting examples of anti-inflammatory agents include celecoxib, rofecoxib, ibuprofen, naproxen, indomethacin, salicylic acid, salsalate, 5-aminosalicylic acid, dimethylfumarate, monomethyl fumarate, methotrexate, predisone, prednisolone, abatecept, aceclofenac, AF-2259, alefacept, amfenac sodium, ampiroxicam, amtolmetin guacil, arformoterol, bambuterol, bardoxolone methyl, butibufen, cankinumab, ciclesonide, deflazacort, doxofylline, dexibuprofen, droxicam, etodolac, flunoxaprofen, fluticasone propionate, fomoterol fumarate, golimumab, indacaterol, interferon-gamma, isofezolac, isoxicam, lobenzarit sodium, lornoxicam, loxoprofen sodium, lumiracoxib, mabuterol HCl, nabumetone, nepafenac, nimesulide, oxaprozin, oxitropium bromide, piroxicam cinnamate, rimexolone, sivelestat, tenoxicam, zaltoprofen, fisalamine, and osalazine.

In some embodiments, the other therapeutic agent is a TNF-α inhibitor. Non-limiting examples of TNF-α inhibitors include infliximab, adalimumab, certolizumab, golimumab, and etanercept.

In some embodiments, the other therapeutic agent is an anti-depressant agent. Non-limiting examples of anti-depressant agents include bupropion HCl, citalopram, desvenlafaxine, fluoxetine HCl, fluvoxamine maleate, metapramine, milnacipran, mirtazapine, moclobemide, nefazodone, paroxetine, pivagabine, reboxetine, setiptiline, sertraline HCl, tianeptine sodium, toloxatone and venlafaxine.

In some embodiments, the other therapeutic agent is an anti-cancer agent. Non-limiting examples of anti-cancer agents include abarelix, alemtuzumab, alitretinoin, amrubicin HCl, amsacrine, anastrozole, arglabin, azacitidine, belotecan, bevacizumab, bexarotene, bicalutamide, bisantrene HCl, bortezomib, camostat mesylate, capecitabine, catumaxomab, cetuximab, cladribine, clofarabine, cytarabine ocfosfate, dasatinib, degarelix acetate, denileukin diftitox, doxetaxel, doxifluridine, enocitabine, epirubicin HCl, erlotinib, exemestane, fludarabine phosphate, flutamide, formestane, fotemustine, fulvestrant, geftimib, gemcitabine HCl, gemtuzumab ozogamicin, ibritumomab tiuxetan, idarubicin HCl, imatibib mesylate, interferon gamma-1a, interleukin-2, irnotecan, INCB18424, ixabepilone, lapatinib, lenalidomide, letrazole, lonidamine, mitoxantrone HCl, nelarabine, nedaplatin, nilutamide, nimotuzumab, OCT-43, ofatumumab, oxaliplatin, paclitaxal, panitumumab, pazopanib, pegaspargase, pemetrexed, pentostatin, pirarubicin, pralatrexate, raltitrexed, ranimustine, ridaforolimus, SKI-2053R, sobuzoxane, sorafenib, sunitinib, talaporfin sodium, tamibarotene, tasonermin, temoporphin, temozolomide, temsirolimus, topotecan HCl, toremifene, tosimomab, trabectedin, valrubicin, vinorelbine, vorinostat and zinostatin stimalamer.

In some embodiments, the other therapeutic agent is an immunosuppressant agent. Non-limiting examples of immunosuppressant agents include cyclosporine, everolimus, gusperimus, mizoribine, muromonab-CD3, mycophenolate sodium, mycophenolate mofeti, pimecrolimus, tacrolimus.

In some embodiments, the other therapeutic agent is an agents to treat osteoporosis. Non-limiting examples of agents to treat osteoporosis include alendronate sodium, ibandronic acid, incadronic acid, raloxifene HCl, risdronate sodium, strontium ranelate.

In some embodiments, the other therapeutic agent is an agent to treat multiple sclerosis. Non-limiting examples of agents to treat multiple sclerosis include dimethyl fumarate, mono methyl fumarate, fingolimod, teriflunomide, laquinimod, cladribine, interferon beta-1a, betaseron, glatimer acetate, natalizumab.

### Methods of Making

Examples of synthetic pathways useful for making Fatty Acid Conjugates of Formula I are set forth in the Examples below and generalized in Schemes 1-9.

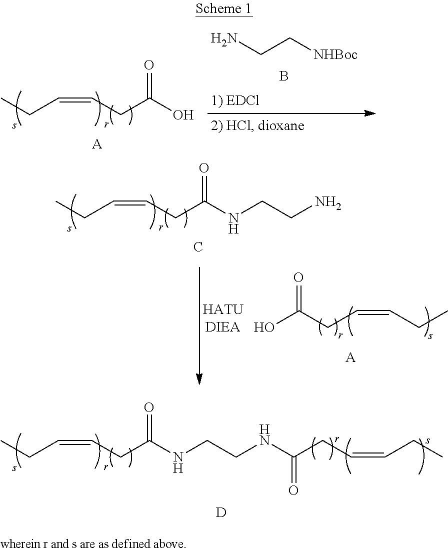

The mono-BOC protected amine of the formula B can be obtained from commercial sources. A fatty acid of formula A can be amidated with the amine B using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound C. To those familiar in the art, the fatty acid A can also be substituted with lipoic acid in this scheme and in the subsequent schemes. Activation of compound C with a coupling agent such as HATU in the presence of an amine such as DIEA followed by addition of a fatty acid of formula C affords compounds of the formula D.

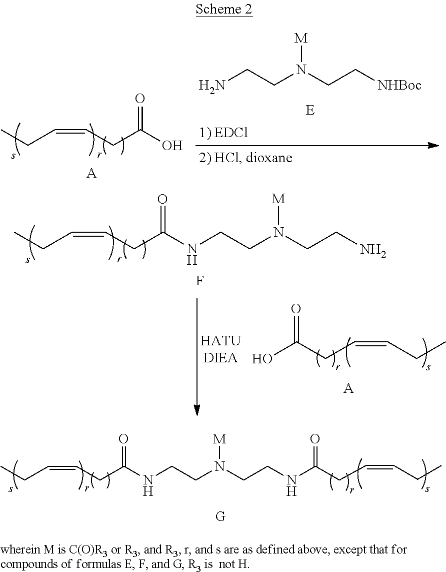

The mono-BOC protected amine of the formula E can be obtained from commercial sources or prepared according to the procedures outlined in Krapcho et al. Synthetic Commun. 1990, 20, 2559-2564 and Andruszkiewicz et al. Synthetic Commun. 2008, 38, 905-913. A fatty acid of formula A can be amidated with the amine E using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound F. Activation of compound F with a coupling agent such as HATU in the presence of an amine such as DIEA followed by addition of a fatty acid of formula A affords compounds of the formula G.

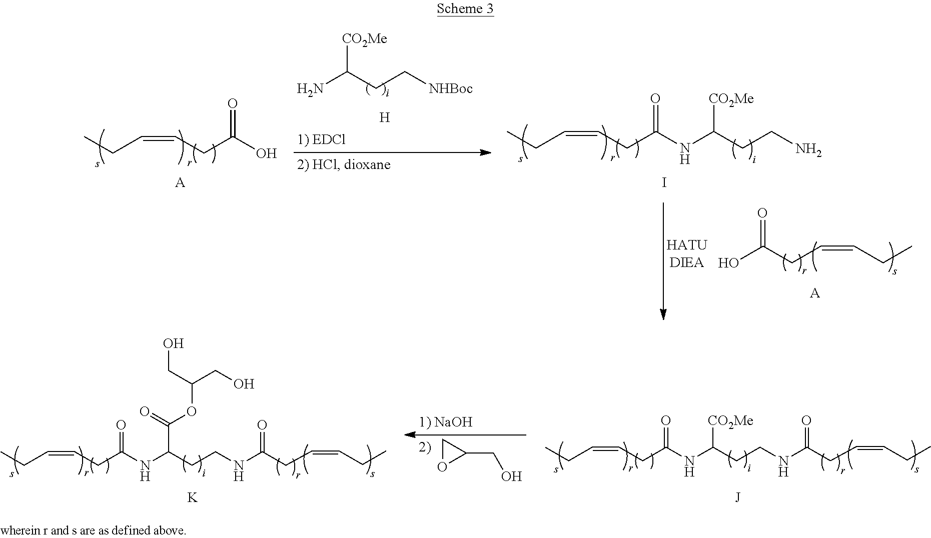

A fatty acid of formula A can be amidated with the corresponding amine H (where i=0, 1, 2 or 3) using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound I. Activation of compound I with a coupling agent such as HATU in the presence of an amine such as DIEA followed by addition of a fatty acid of formula A affords compounds of the formula J. Hydrolysis of the ester under basic conditions such as NaOH or LiOH produces the corresponding acid, which can be coupled with glycidol to afford compounds of the formula K.

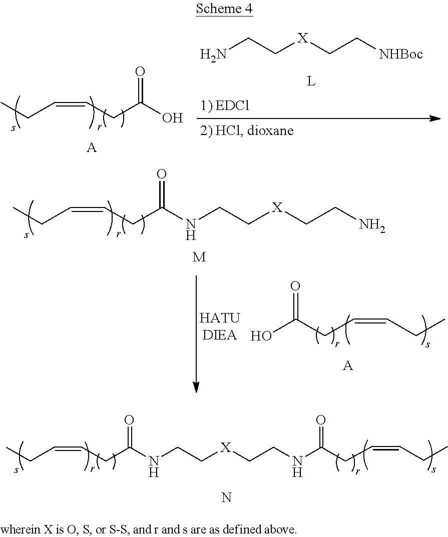

The amine L can be obtained from commercial sources or prepared according to the procedures outlined in Dahan et al. J. Org. Chem. 2007, 72, 2289-2296 and Jacobson, K. et al. Bioconjugate Chem. 1995, 6, 255-263. A fatty acid of formula A can be coupled with the amine L using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound M. Activation of compound M with a coupling agent such as HATU in the presence of an amine such as DIEA followed by addition of a fatty acid of formula A affords compounds of the formula N. To those skilled in the art, the sulfur group in formula N (when X═S) can be oxidized to the corresponding sulfoxide or sulfone using an oxidizing agent such as H₂O₂ or oxone.

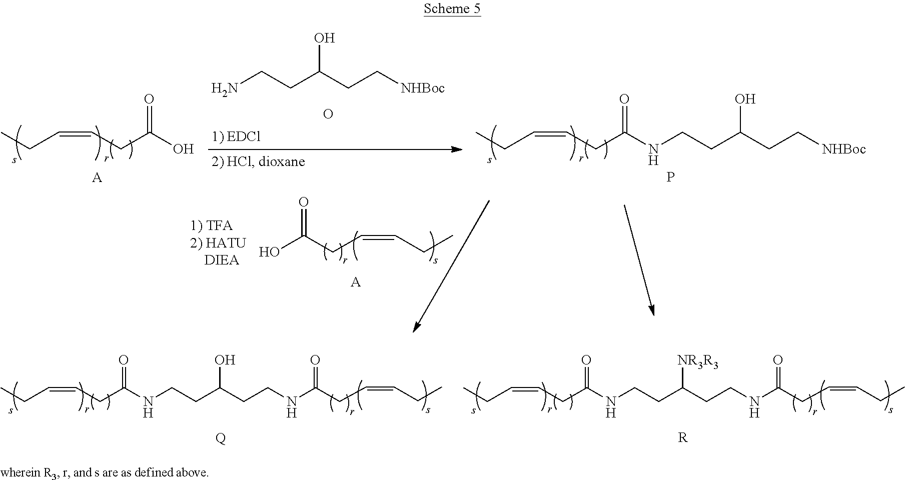

The amine 0 can be prepared from the commercially available diamine according to the procedures outlined in Dahan et al. J. Org. Chem. 2007, 72, 2289-2296. Fatty acids of the formula A can be amidated with the amine 0 using a coupling reagent such as DCC, CDI, EDC, or optionally with a tertiary amine base and/or catalyst, e.g., DMAP, to afford compound P. The BOC group of compound P can be removed with acids such as TFA or HCl in a solvent such as CH₂Cl₂ or dioxane and the resulting amine can be coupled with a fatty acid of formula A using HATU in the presence of an amine such as DIEA to afford compounds of the formula Q. To those skilled in the art, the hydroxyl group in compound P can be further acylated or converted to an amino group by standard mesylation chemistry followed by displacement with sodium azide and reduction with an agent such as triphenylphosphine. The amine can be further acylated or alkylated, followed by the removal of the BOC group. The resulting amine can be coupled with a fatty acid of the formula A to afford compounds of the formula R.

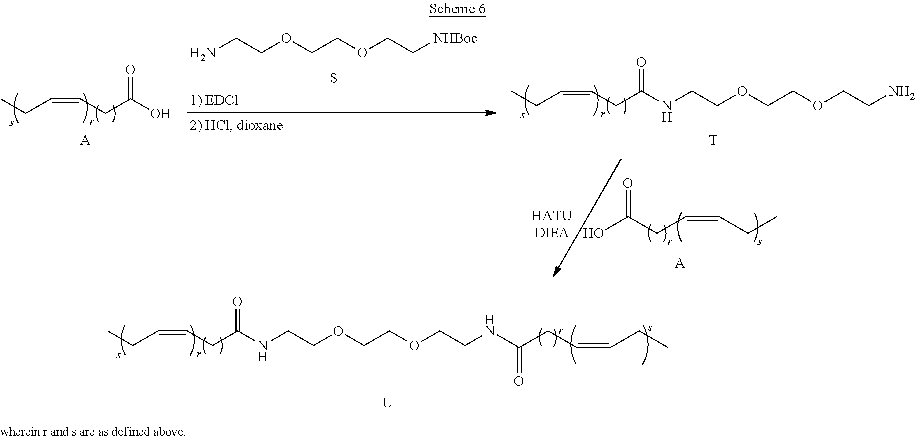

A fatty acid of formula A can be amidated with the commercially available amine S using a coupling reagent such as DCC, CDI, EDC, optionally with a tertiary amine base and/or catalyst, e.g., DMAP. The BOC group of the resulting coupled product can be removed with acids such as TFA or HCl in a solvent such as CH₂Cl₂ or dioxane to afford compound T. The resulting amine can be coupled with a fatty acid of the formula A using a coupling agent such as HATU in the presence of an amine such as DIEA to afford compounds of the formula U.

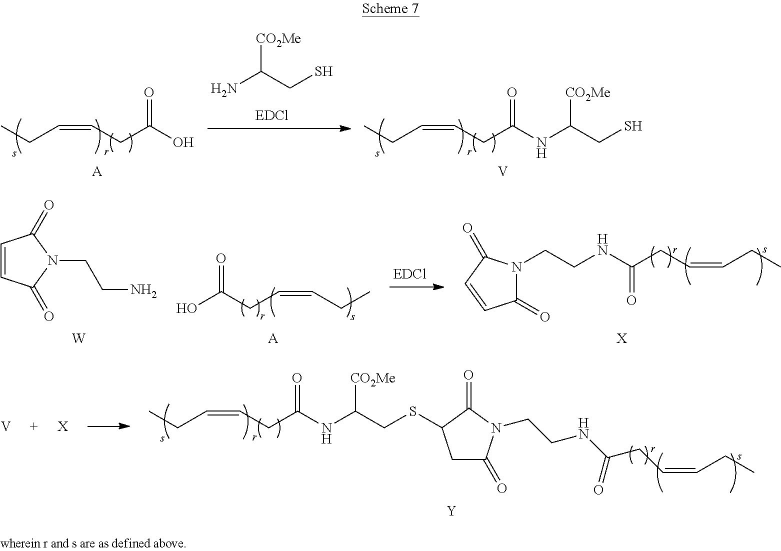

A fatty acid of formula A can be amidated with the commercially available cysteine methyl ester using a coupling reagent such as DCC, CDI, EDC, or optionally with a tertiary amine base and/or catalyst, e.g., DMAP, to afford compound V. The commercially available maleimide derivative W can be coupled with a fatty acid of the formula A using a coupling agent such as HATU or EDCI to afford compounds of the formula X. Compound V can be coupled to compounds of the formula X in a solvent such as acetonitrile to afford compounds of the formula Y.

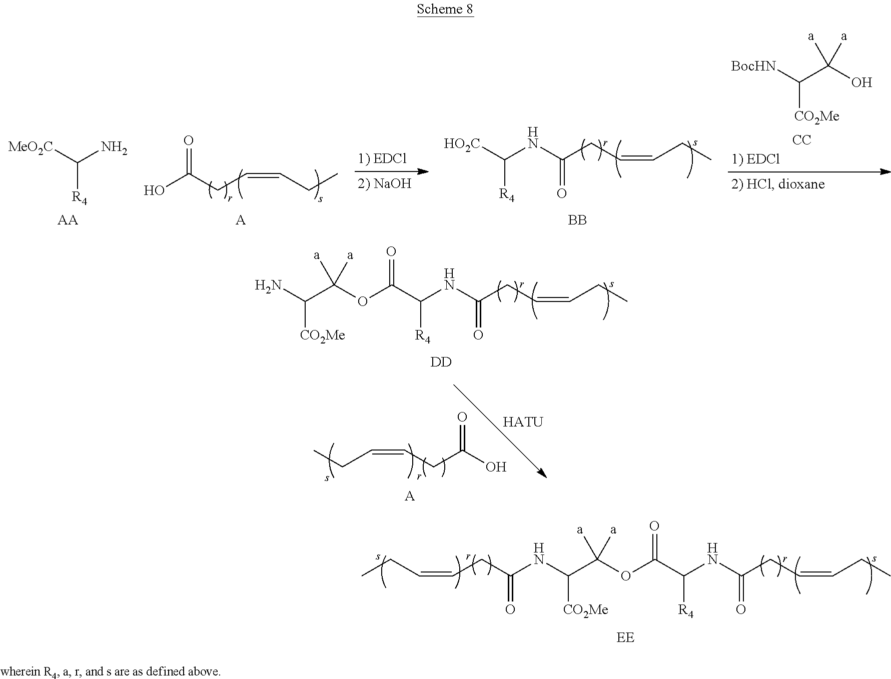

Commercially available amino acid esters AA can be coupled with a fatty acid of the formula A using a coupling agent such as EDCI or HATU, followed by alkaline hydrolysis of the methyl ester to afford compounds of the formula BB. Compounds of the formula BB can be coupled with the commercially available BOC-amino acid derivatives CC using a coupling agent such as EDCI or HATU. The BOC group can be removed by treatment with acids such as TFA or HCl to afford compounds of the formula DD which can then be coupled with a fatty acid of formula A to afford compounds of the formula EE.

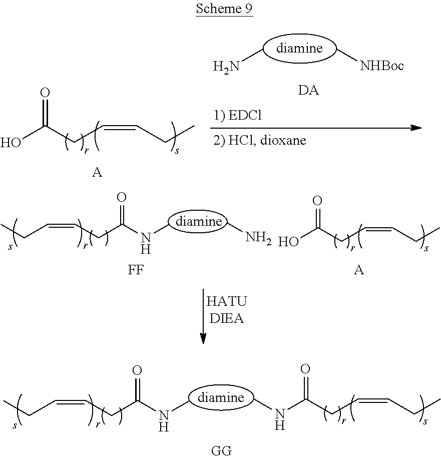

A fatty acid of formula A can be coupled with a BOC-protected diamine of the general formula DA to obtain the BOC-protected amide derivative. After treatment with HCl in dioxane, the resulting amine FF can be coupled with a fatty acid of the formula A in order to obtain compounds of the formula GG. A variety of BOC-protected diamines are commercially available. The following diamines can be prepared according to the procedures outlined in the corresponding references:

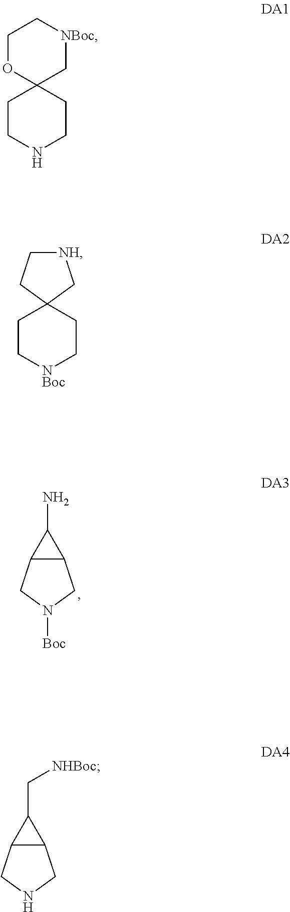 [SMILES: CC(C)(C)OC(=O)N1CCOC2(CCNCC2)C1 ; CC(C)(C)OC(=O)N1CCC2(CCNC2)CC1 ; CC(C)(C)OC(=O)N1CC2C(N)C2C1 ; CC(C)(C)OC(=O)NCC1C2CNCC21]

diamine DA1, Stocks et al, Bioorganic and Medicinal Chemistry Letters 2010, p. 7458;
diamine DA2, Fritch et al, Bioorganic and Medicinal Chemistry Letters 2010, p. 6375;
diamine DA3 and DA4, Moffat et al, J. Med. Chem. 2010, 53, p. 8663-86′78). To those familiar in the art, detailed procedures to prepare a variety of mono-protected diamines can also be found in the following references: WO 2004092172, WO 2004092171, and WO 2004092173.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

### Effect of Bis-Fatty Acid Conjugates on ApoB Secretion in HepG2 Cells

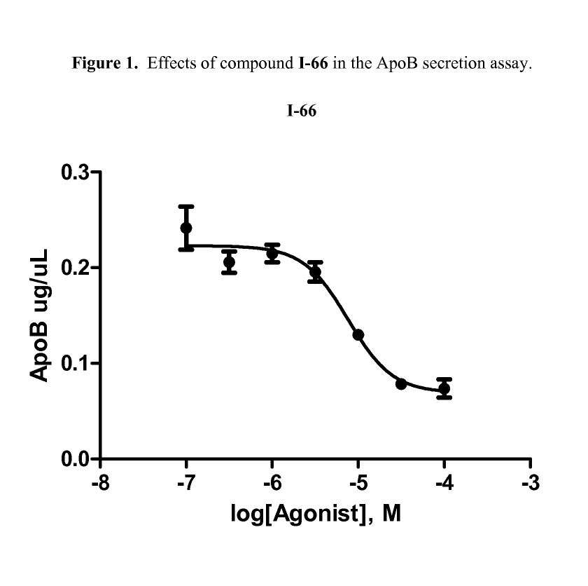

HepG2 cells (ATCC) are seeded at 25,000 cells per well in collagen-coated 96-well plates in growth media (DMEM with 10% fetal bovine serum). The following day, bis-fatty acid conjugates are complexed to lipoprotein-deficient fetal bovine serum at the appropriate concentration. Growth media is then removed from and the HepG2 cells are washed once with PBS. The lipoprotein-deficient FBS with the complexed bis-fatty acid conjugates is added to DMEM for a final 10% concentration. Each concentration of bis-fatty acid is tested in triplicate. Cells are incubated for 16 hours with the bis-fatty acid. Alamar Blue® (Invitrogen) is then added to the media to determine cell viability per the manufacturer's instructions. Two hours after Alamar Blue® addition, the media is removed and placed in a black 96-well plate. The plate is then read at 550 nm/590 nm to determine cell viability. The media is then used to determine ApoB concentrations using ELISA kits (Mabtech AB). Percent inhibition of ApoB secretion is determined by normalizing data to vehicle treated wells. For a given compound, an IC₅₀ (concentration at which 50% of ApoB secretion is inhibited) can also be determined by using a 4 parameter-fit inhibition curve model (Graph Pad Prism®). As an illustrative example, FIG. 1 shows the effects of compound I-66 in the ApoB assay. The IC₅₀ was determined to be 9.5 μM.

### Example 2

### Effect of Bis-Fatty Acid Conjugates on SREBP-1c Target Genes

HepG2 cells (ATCC) are seeded at 20,000 cells per well in 96 well plates. After adhering overnight, growth media (10% FBS in DMEM) is removed and cells are serum starved for 24 hours in DMEM containing 1% fatty acid free bovine serum albumin (BSA, Sigma). Cells are then treated with the bis-fatty acid conjugates at a final concentration of 50 μM in 1% BSA or 0.1 oleate complexed to fatty acid free BSA in a 5:1 molar ratio. Cells are incubated for 6 hours and then washed with PBS. RNA was reverse-transcribed using the cells II cDNA reagents according to standard protocols (outlined in Applied Biosystem StepOne Real-time PCR protocols). Real time PCR of transcripts can be performed with Tagman assays for the three specific genes FASN (fatty acid synthase), SCD (steroyl CoA desaturase) and ApoAl(apolipoprotein Al). In all three cases, 185-VIC® is used as a normalization control.

### Example 3

### Effect of Bis-Fatty Acid Conjugates in the Zucker fa/fa Rat Model

Male Zucker rats (HsdHlr:ZUCKER-Lepr̂fa) between 8-10 weeks of age are purchased from Harlan. Zucker rats are maintained on Teklad Global Rodent Diet (2018S) during the acclimation period and for the duration of study. The Zucker rats are weighed and randomly assigned to treatment arms based on body weight and plasma TG levels (n=8). Inclusion criteria for the study include body weight >300 grams and fed TG levels in plasma >800 mg/dL. Rats are randomized into treatment arms based on pre-dose (day -1) body weights and plasma levels (fed) of triglycerides. Dosing is initiated on day 1 and continue through day 5. Dosing is daily (qd) by oral gavage (po) for all treatment arms. Body weights are measured for all rats on days 1 through 5. On day 4, a blood sample (fed) are collected from each rat, processed for plasma and stored at −80° C. At 8 pm on day 4 food are removed from all rats to initiate fasting state. On day 5 rats are dosed at 8 am according to treatment arm. Two hours later (10 am) two blood draws from each rat are collected and processed for plasma. Triglyceride levels are then analyzed by standard protocols using commercially available kits.

### Example 4

### Effect of Bis-Fatty Acid Conjugates in the Golden Syrian Hamster Model of Dyslipidemia

Golden Syrian Hamster (Strain: HsdHan™:AURA, from Harlan Laboratories), 5-6 weeks of age, with a body weight of approximately 80 g, are used for the study. The Hamsters are maintained on high fat diet D12492 (Research Diets, New Brunswick N.J.) during the acclimation period and throughout the study. Animals will then receive drinking water supplemented with 10% fructose (Sigma, supplied by Catabasis) starting on day -8 and continuing throughout the study. The hamsters will be randomized into treatment arms based on pre-dose (day -1) body weights and plasma levels (fed) of triglycerides (TG). Dosing will be initiated on day 1 and continue through day 28. Dosing will be daily (qd) by oral gavage (po) for all treatment arms. On day 27, hamsters will be fasted at the beginning of the dark cycle. Hamsters will be dosed at 8 am on day 28 according to the treatment arm. Two hours later (10 am) a blood sample will be collected from each hamster, processed to plasma and stored at −80oC. Triglyceride and HDL cholesterol levels will be determined using standard protocols and the commercially available kits from Abcam, Cayman or Sigma-Aldrich.

### Example 5

### Effects of compounds of the invention on NFκB Levels in RAW 264.7 Macrophages

RAW 264.7 cells stably expressing a 3× NFkB response elemement-drive luciferase reporter were seeded into 96 well plates in sera-free medium (Optimem) 18 hours prior to compound application. Compounds of the invention were prepared by first making 100 mM stock solutions in EtOH. Stock solutions were then diluted 1:100 in low LPS FBS (Gemini BenchMark 100-106), mixed vigorously and allowed to incubate at room temperature for 30 minutes. 1:2 serial dilutions were then made in FBS supplemented with 1% EtOH, mixed vigorously, and again allowed to incubate at room temperature for 30 minutes before adding to RAW 264.7 reporter cells (final concentrations: 10% FBS, 100 uM highest compound dilution, 0.1% EtOH) for a 2 hour pretreatment prior to stimulation with LPS. Cells were then stimulated with 200 ng/ml LPS or vehicle control for 3 hours in the presence of the compounds of the invention. A set of six vehicles was left unstimulated with LPS in order to measure the assay floor. AlamarBlue viability dye (Invitrogen) was added to cells simultaneously with the delivery of LPS (final AlamarBlue concentration of 10%).

After the 3 h incubation period with LPS, cell viability was measured by reading fluorescence (excitation 550 nm, emission 595 nm) with a Perkin Elmer Victor V plate reader. Then cell media was aspirated from each well. Luciferase signal was then developed by addition of the Britelite Plus reagent (Perkin Elmer). Luciferase activity was measured with the Perkin Elmer Victor V plate reader. NF-κB activity was expressed as a percent of the vehicle control wells (stimulated with LPS). Compounds were tested at 6 dose point titrations in triplicate to determine IC₅₀ values. Table 1 summarizes the 1050 values for a number of bis-fatty acid conjugates in this NF-κB luciferase reporter assay. A (−) indicates that the compound showed no inhibitory activity up to 200 μM. A (+) indicates that the compound showed inhibitory activity of less than 200 μM. A (++) indicates that the compound showed inhibitory activity of less than 50 μM.

TABLE 1

| | NF-kB inhibitory |
|---|---|
| Compound | activity IC₅₀ μM |
| DHA | − |
| EPA | − |
| I-1 | + |
| I-2 | ++ |
| I-37 | + |
| I-39 | ++ |
| I-45 | ++ |
| I-65 | ++ |
| I-66 | ++ |
| I-67 | + |
| I-107 | + |

### Example 6

### Effect of bis-fatty acid conjugates on IL-1β

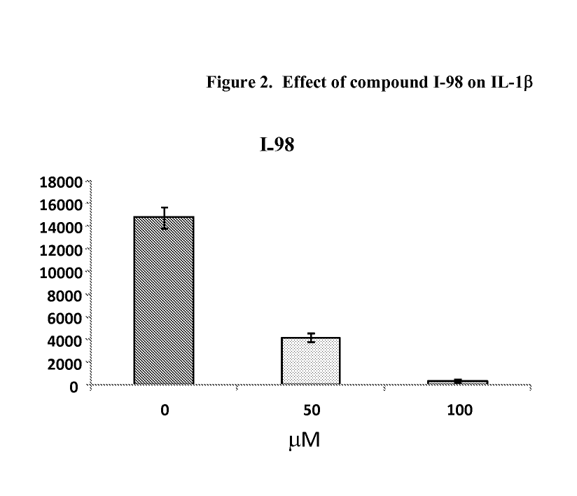

RAW264.7 macrophages were seeded at a density of 100,000 cells/well in a 96-well plate in DMEM supplemented with 10% FBS and Penn/strep. 16 hours later, medium was aspirated and replaced with 904/well of serum-free DMEM. Bis-fatty acid conjugates were brought up in 100% EtOH to a concentration of 100 mM and then diluted 1:100 in 100% FBS for a stock solution consisting of 1 mM compound and 1% EtOH. These stock solutions were then diluted 1:10 in FBS supplemented with 1% EtOH to generate a 100 μM of the bis-fatty acid conjugate. 10 μL was then added to the RAW246.7 cells to generate final concentrations 10 μM of the bis-fatty acid conjugate, along with vehicle only control. The compounds were allowed to pre-incubate for 2 hours before stimulation of 100 ng/ml LPS (10 μL of 1 μg/ml LPS was added to each well). Following 3 hours of LPS stimulation, cells were washed once in 1×PBS, aspirated dry, and flash frozen in liquid nitrogen. RNA was then isolated and converted to cDNA using the Cells to cDNA kit (Ambion) according to the manufacturer's protocol. IL-1β transcript levels were then measured using Taqman primer/probe assay sets (Applied Biosystems), normalized to GAPDH using the deltaCt method, and the data expressed relative to vehicle only control. As an illustrative example, FIG. 2 shows the decrease in IL-1β gene expression when compound I-98 was dosed at 0, 50 and 100 μM.

### Example 7

### Effect of Bis-Fatty Acid Conjugates on the Target Gene Hmox1 in RAW Macrophages

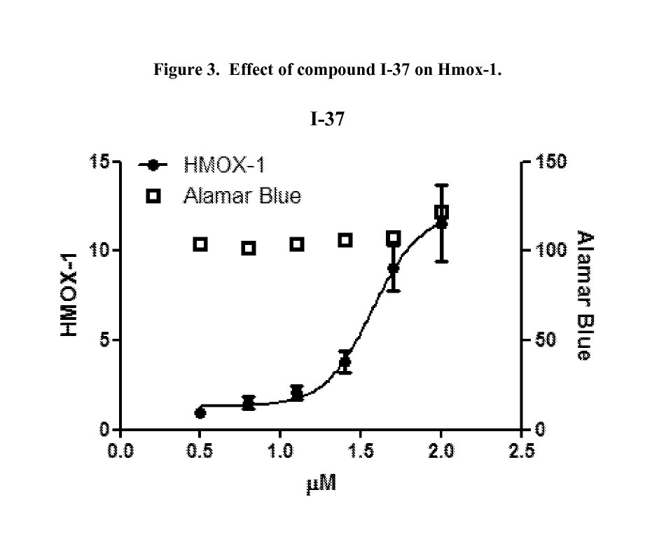

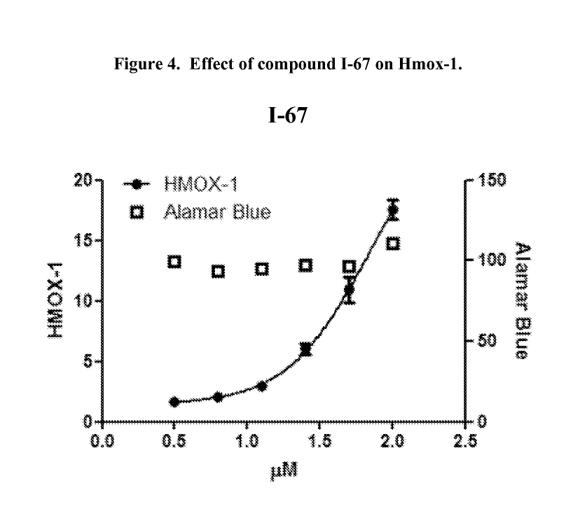

RAW264.7 macrophages were seeded at a density of 100,000 cells/well in a 96-well plate in DMEM supplemented with 10% FBS and Penn/strep. 16 hours later, medium was aspirated and replaced with 90 uL/well of serum-free DMEM. Bis-fatty acid conjugates were brought up in 100% EtOH to a concentration of 100 mM and then diluted 1:100 in 100% FBS for a 20× stock solution consisting of 1 mM compound and 1% EtOH. The bis-fatty acid conjugate 20× stock solutions were diluted 1:2 in FBS supplemented with 1% EtOH for a 500 uM 10× stock solution. The 10× stock solutions were then serially diluted 1:2 in FBS supplemented with 1% EtOH and 104, of each dilution was added to the RAW246.7 cells to generate final concentrations of 50, 25, 12.5, 6.25, 3.12 and 1.6 μM. The compounds were allowed to pre-incubate for 2 hours before stimulation of 100 ng/mlLPS (104 of 1 μg/ml LPS is added to each well). Following 3 hours of LPS stimulation, cells were washed once in 1×PBS, aspirated dry, and flash frozen in liquid nitrogen. RNA was then isolated and converted to cDNA using the Cells to cDNA kit (Ambion) according to the manufacturer's protocol. Transcript levels were then measured using ABI Taqman primer/probe assay kits, normalized to GAPDH using the deltaCt method, and the data expressed relative to vehicle only control. As illustrative examples, FIGS. 3 and 4 show the effect of compounds I-37 and 1-67 on Hmox-1.

### Example 8

Effect Of Bis-Fatty Acid Conjugates in the Streptozotocin-Diabetic Rat

Female Sprague-Dawley rats (8 weeks old, with an average weight of 150 g) are used for the study. Diabetes is induced by a single tail vein injection of streptozotocin (STZ) in 0.1 mol/L sodium citrate buffer, pH 4.5. Diabetes is then confirmed by measuring blood glucose levels at two and three days after the STZ treatment. Diabetic animals are classified as those with plasma glucose higher than 16 nmol/L. The diabetic animals are then divided into the vehicle control group and the treatment group (each group having 12 animals). All animals are housed individually with a light dark cycle of 12 hours each, with animals having free access to food and water. In order to maintain body weight and to limit hyperglycemia, diabetic animals are treated with 3 IU of ultralente insulin three times per week in the afternoon (at approximately 3 to 4 pm). In order to maintain glycemic control as the animals gain weight, the dose of insulin is increased to 5 IU at week 15. Animals are dosed with the vehicle or the bis-fatty acid conjugate over a 28 week period (Examples of vehicles that can be used include combinations of solvents such as polyethylene glycol and propyleneglycol, lipids such as glycerol monooleate and soybean oil, and surfactants such as polysorbate 80 and cremophor EL). Progression of renal disease can be assessed by monthly measurements of urinary albumin and plasma creatinine concentrations. For urinary measurements, rats are housed in metabolic rat cages for 24 hrs. Urinary albumin can be quantified by a competitive ELISA assay according to the protocols outlined in Degenhardt et al, Kidney International 2002, 61, p. 939-950. Plasma creatinine concentrations can be measured by the Jaffe picric acid procedure, using the standard kit from Sigma (Sigma cat #555-A). Statistical analyses can be performed using SigmaStat for Windows V1.00. P values can be calculated by non-parametric Mann-Whitney Rank Sum analysis. On week 28, dyslipidemia can also be assessed by measuring plasma triglycerides and total cholesterol. These plasma lipids can be measured by enzymatic, colorimetric, end-point assays using standardized, commercially available kits. Total cholesterol can be analyzed using the Sigma kit (cat #352) and triglycerides can be analyzed by the Sigma kit (cat #37, GOP Grinder).

### Example 9

### Effect of bis-fatty acid conjugates in the cisplatin-induced nephrotoxicity mouse model

For this study, 10 to 12-week old male C57BL/6 mice of approximately 30 g in body weight are used. After the normal acclimation period, the animals are maintained on a standard diet and water is freely available. Mice are then given a single intraperitoneal injection of either the vehicle or cisplatin (20 mg/kg, at a concentration of 1 mg/mL in saline). Ten animals are used per treatment group. For the drug treatment group, beginning 24 hours prior to the cisplatin injection, animals are dosed with a bis-fatty acid conjugate (formulated in combinations of solvents such as polyethylene glycol and propyleneglycol, lipids such as glycerol monooleate and soybean oil, and surfactants such as polysorbate 80 and cremophor EL). Dosing is then continued over a period of 72 hours. At this point, animals are sacrificed and blood and kidney tissues are collected. Blood urea nitrogen (BUN) and creatinine are measured. Levels of TNF-α in serum can be determined using a commercially available enzyme-linked immunosorbent assay (ELISA). Tissues are processed for histology and RNA isolation. Tubular injury can be assessed in PAS-stained sections using a semi-quantitative scale described in “G. Ramesh and W. B. Reeves, Kidney International, 2004, 65, p. 490-498”.

The following non-limiting compound examples serve to illustrate further embodiments of the bis-fatty acid conjugates. It is to be understood that any embodiments listed in the Examples section are embodiments of the bis-fatty acid conjugates and, as such, are suitable for use in the methods and compositions described above.

### Example 10

### Preparation of (4Z,7Z,10Z,13Z,16Z,19Z)-docosa-4,7,10,13,16,19-hexaenoic acid [2-((5Z,8Z,11Z,14Z,17Z)-icosa-5,8,11,14,17-pentaenoylamino)-ethyl]-amide (I-37)

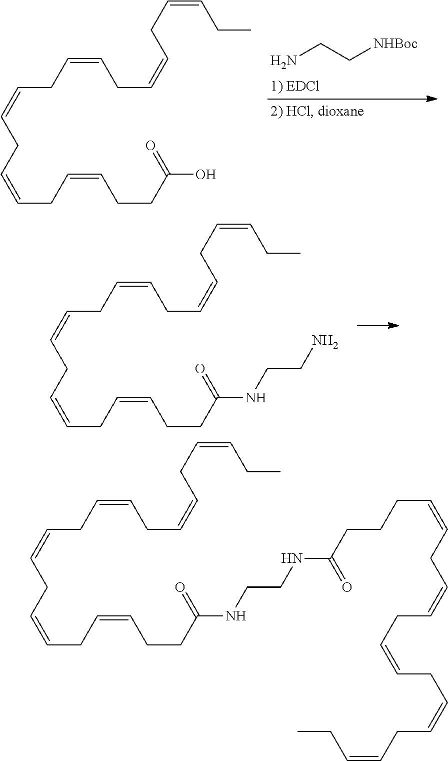

(4Z,7Z,10Z,13Z,16Z,19Z)-docosa-4,7,10,13,16,19-hexaenoic acid (1 mmol) was taken up in CH₃CN (5 mL) along with tert-butyl 2-aminoethylcarbamate (1 mmol) and EDCI (1.1 mmol). The resulting reaction mixture was stirred at room temperature for 2 h. It was then washed with brine, dried over Na₂SO₄, filtered and concentrated under reduced pressure. Purification by silica gel chromatography (CH₂Cl₂) afforded tert-butyl 2-(4Z,7Z,10Z,13Z,16Z,19Z)-docosa-4,7,10,13,16,19-hexaenamidoethylcarbamate.

tert-Butyl 2-(4Z,7Z,10Z,13Z,16Z,19Z)-docosa-4,7,10,13,16,19-hexaenamidoethylcarbamate (500 mg, 1.06 mmol) was taken up in 4M HCl in dioxane (3 mL). The resulting reaction mixture was allowed to stir at room temperature for 10 min. It was then diluted with EtOAc (10 mL) and concentrated under reduced pressure to afford the HCl salt of (4Z,7Z,10Z,13Z,16Z,19Z)-N-(2-aminoethyl)docosa-4,7,10,13,16,19-hexaenamide.

This HCl salt of (4Z,7Z,10Z,13Z,16Z,19Z)-N-(2-aminoethyl)docosa-4,7,10,13,16,19-hexaenamide (1.06 mmol) was taken up in CH₃CN (5 mL) along with (5Z,8Z,11Z,14Z,17Z)-eicosa-5,8,11,14,17-pentaenoic acid (0.35 mmol), HATU (0.55 mmol) and DIEA (1.5 mmol). The resulting reaction mixture was stirred at room temperature for 1 h and concentrated under reduced pressure. The resulting residue was purified by silica gel chromatography (95% CH₂Cl₂, 5% MeOH) to afford 530 mg of (4Z,7Z,10Z,13Z,16Z,19Z)-docosa-4,7,10,13,16,19-hexaenoic acid [2-((5Z,8Z,11Z,14Z,17Z)-icosa-5,8,11,14,17-pentaenoylamino)-ethyl]-amide (81% yield). MS (EI) calcd for C₄₄H₆₆N₂O₂: 654.51. found 655 (M+1).

### Example 11

### Preparation of (4Z,7Z,10Z,13Z,16Z,19Z)-docosa-4,7,10,13,16,19-hexaenoic acid [2-((4Z,7Z,10Z,13Z,16Z,19Z)-docosa-4,7,10,13,16,19-hexaenoylamino)-ethyl]-amide (I-1)

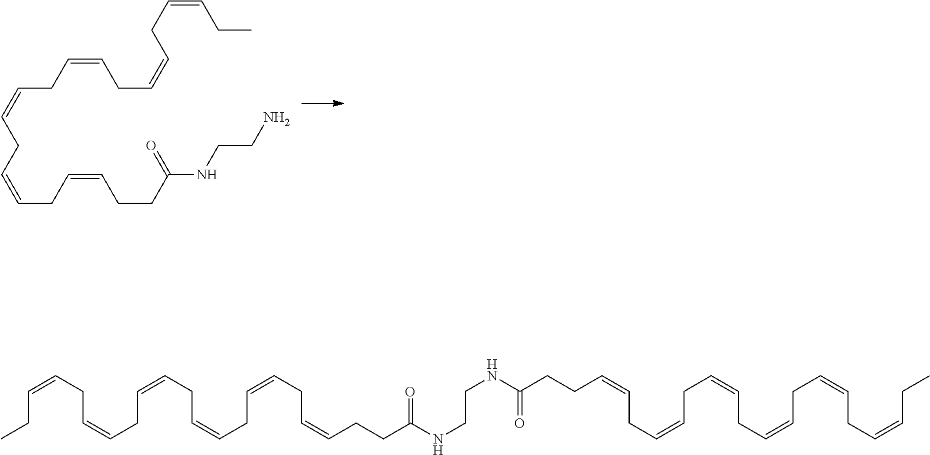 [SMILES: CC/C=C\C/C=C\C/C=C\C/C=C\C/C=C\C/C=C\CCC(=O)NCCN ; CC/C=C\C/C=C\C/C=C\C/C=C\C/C=C\C/C=C\CCC(=O)NCCNC(=O)CC/C=C\C/C=C\C/C=C\C/C=C\C/C=C\C/C=C\CC]

The same experimental procedure detailed above for the preparation of (4Z,7Z,10Z,13Z,16Z,19Z)-docosa-4,7,10,13,16,19-hexaenoic acid [2-((5Z,8Z,11Z,14Z,17Z)-icosa-5,8,11,14,17-pentaenoylamino)-ethyl]-amide was used, substituting (4Z,7Z,10Z,13Z,16Z,19Z)-docosa-4,7,10,13,16,19-hexaenoic acid for (5Z,8Z,11Z,14Z,17Z)-eicosa-5,8,11,14,17-pentaenoic acid in the last amide coupling step. MS (EI) calcd for C₄₆H₆₈N₂O₂: 680.53. found 681 (M+1).

### Example 12

### Preparation of (4Z,7Z,10Z,13Z,16Z,19Z)-docosa-4,7,10,13,16,19-hexaenoic acid [2-((R)-5-[1,2]dithiolan-3-yl-pentanoylamino)-ethyl]-amide (I-98)

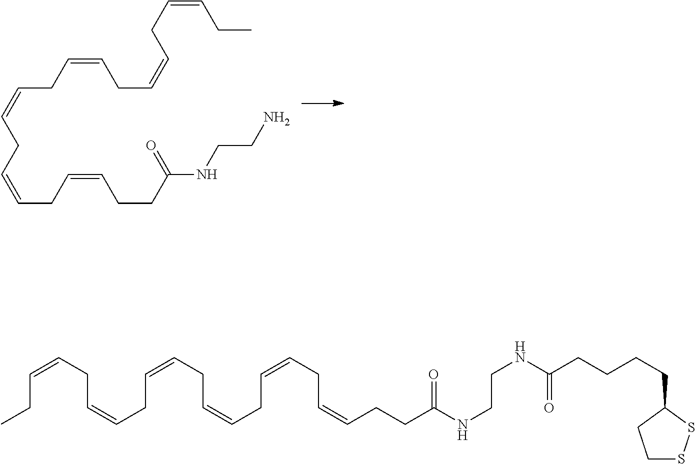 [SMILES: CC/C=C\C/C=C\C/C=C\C/C=C\C/C=C\C/C=C\CCC(=O)NCCN ; CC/C=C\C/C=C\C/C=C\C/C=C\C/C=C\C/C=C\CCC(=O)NCCNC(=O)CCCC[C@@H]1CCSS1]

The same experimental procedure detailed above for the preparation of (4Z,7Z,10Z,13Z,16Z,19Z)-docosa-4,7,10,13,16,19-hexaenoic acid [2-((5Z,8Z,11Z,14Z,17Z)-icosa-5,8,11,14,17-pentaenoylamino)-ethyl]-amide was used, substituting R-α-lipoic acid for for (5Z,8Z,11Z,14Z,17Z)-eicosa-5,8,11,14,17-pentaenoic acid in the last amide coupling step. MS (EI) calcd for C₃₄H₅₅N₃O₂S₂: 601.37. found 602 (M+1).

### Example 13

### Preparation of (4Z,7Z,10Z,13Z,16Z,19Z)-docosa-4,7,10,13,16,19-hexaenoic acid (2-{[2((5Z,8Z,11Z,14Z,17Z)-icosa-5,8,11,14,17-pentaenoylamino)-ethyl]-methyl-amino}-ethyl)-amide (I-39)

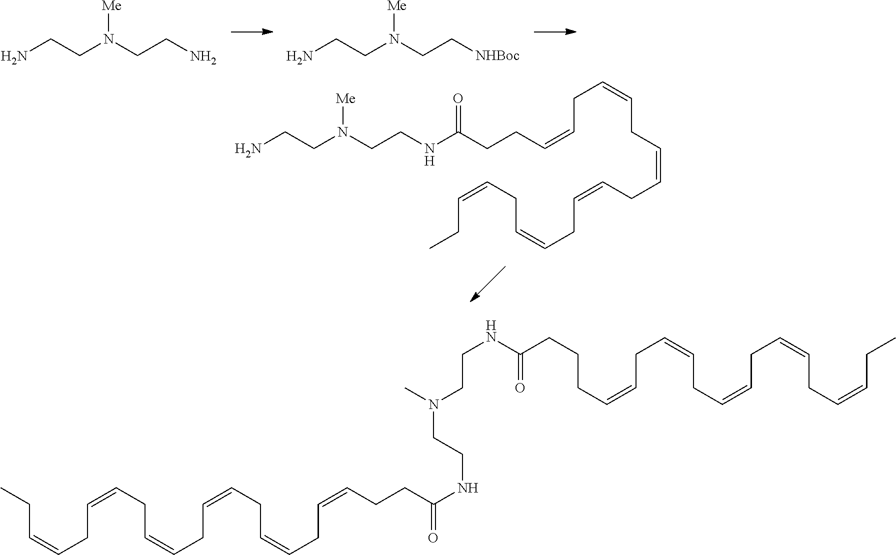 [SMILES: CN(CCN)CCN ; CN(CCN)CCNC(=O)OC(C)(C)C ; CC/C=C\C/C=C\C/C=C\C/C=C\C/C=C\C/C=C\CCC(=O)NCCN(C)CCN ; CC/C=C\C/C=C\C/C=C\C/C=C\C/C=C\C/C=C\CCC(=O)NCCN(C)CCNC(=O)CCC/C=C\C/C=C\C/C=C\C/C=C\C/C=C\CC]

N1-(2-Aminoethyl)-N-1-methylethane-1,2-diamine (5.0 g, 42.7 mmol) was dissolved in 100 mL of CH₂Cl₂ and cooled to 0° C. A solution of di-tert-butylcarbonate (0.93 g, 4.27 mmol) in CH₂Cl₂ (10 mL) was then added dropwise at 0° C. over a period of 15 min. The resulting reaction mixture was stirred at 0° C. for 30 min and then warmed to room temperature. After stirring at room temperature for 2 h, the reaction mixture was diluted with CH₂Cl₂ (100 mL). The organic layer was washed with brine (3×25 mL), dried (Na₂SO₄) and concentrated under reduced pressure to afford 1.1 g of tert-butyl 2-((2-aminoethyl)(methyl)amino)ethylcarbamate.

tert-butyl 2-((2-aminoethyl)(methyl)amino)ethylcarbamate (430 mg, 1.98 mmol) was taken up in 10 mL of CH₃CN along with (4Z,7Z,10Z,13Z,16Z,19Z)-docosa-4,7,10,13,16,19-hexaenoic acid (DHA, 650 mg, 1.98 mmol), HATU (750 mg, 2.2 mmol) and DIEA (0.550 mL). The resulting reaction mixture was stirred at room temperature for 2 h and then diluted with EtOAc (40 mL). The organic layer was washed with brine, dried (Na₂SO₄) and concentrated under reduced pressure. Purification by chromatography (95% CH₂Cl₂, 5% MeOH) afforded 400 mg of the Boc-protected intermediate. This material was taken up in 3 mL of 4 M HCl in dioxane and allowed to stir at room temperature for 10 min.

The reaction mixture was concentrated under reduced pressure to afford the HCl salt of (4Z,7Z,10Z,13Z,16Z,19Z)-N-(2-((2-aminoethyl)(methyl)amino)ethyl)docosa-4,7,10,13,16,19-hexaenamide.

This HCl salt of (4Z,7Z,10Z,13Z,16Z,19Z)-N-(2-((2-aminoethyl)(methyl)amino)ethyl)docosa-4,7,10,13,16,19-hexaenamide (0.38 mmol) was taken up in 4 mL of CH₃CN along with EPA (114 mg, 0.38 mmol), HATU (160 mg, 0.42 mmol) and DIEA (75 μL, 1.14 mmol). The resulting reaction mixture was stirred at room temperature for 2 h and diluted with EtOAc (25 mL). The organic layer was washed with brine, dried (Na₂SO₄) and concentrated under reduced pressure. Purification by chromatography (95% CH₂Cl₂, 5% MeOH) afforded 200 mg of (4Z,7Z,10Z,13Z,16Z,19Z)-docosa-4,7,10,13,16,19-hexaenoic acid (2-{[2((5Z,8Z,11Z,14Z,17Z)-icosa-5,8,11,14,17-pentaenoylamino)-ethyl]-methyl-amino}-ethyl)-amide. MS (EI) calcd for C₄₇H₇₃N₃O₂: 711.57. found 712 (M+1).

### Example 14

### Preparation of (2S,3R)-methyl 3-((S)-2-((4Z,7Z,10Z,13Z,16Z,19Z)-docosa-4,7,10,13,16,19-hexaenamido)propanoyloxy)-2-((5Z,8Z,11Z,14Z,17Z)-icosa-5,8,11,14,17-pentaenamido)butanoate (I-66)

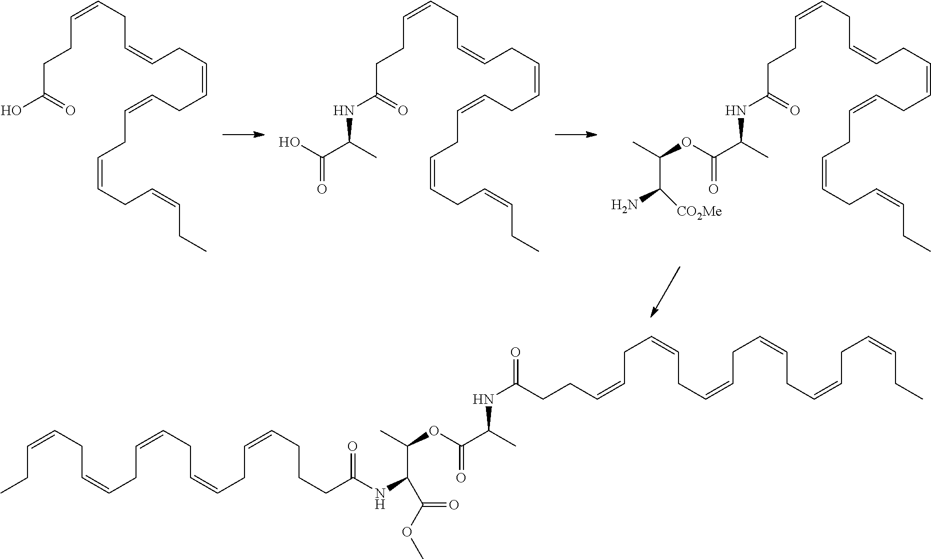 [SMILES: CC/C=C\C/C=C\C/C=C\C/C=C\C/C=C\C/C=C\CCC(=O)O ; CC/C=C\C/C=C\C/C=C\C/C=C\C/C=C\C/C=C\CCC(=O)N[C@@H](C)C(=O)O ; CC/C=C\C/C=C\C/C=C\C/C=C\C/C=C\C/C=C\CCC(=O)N[C@@H](C)C(=O)O[C@H](C)[C@H](N)C(=O)OC ; CC/C=C\C/C=C\C/C=C\C/C=C\C/C=C\C/C=C\CCC(=O)N[C@@H](C)C(=O)O[C@H](C)[C@H](NC(=O)CCC/C=C\C/C=C\C/C=C\C/C=C\C/C=C\CC)C(=O)OC]

(4Z,7Z,10Z,13Z,16Z,19Z)-docosa-4,7,10,13,16,19-hexaenoic acid (2 mmol) was taken up in CH₃CN (10 mL) along with L-alanine methyl ester (2 mmol) and EDCI (2.2 mmol). The resulting reaction mixture was stirred at room temperature for 2 h and diluted with EtOAc. The organic layer was washed with dilute aqueous NaHCO₃, brine, dried over Na₂SO₄, filtered and concentrated under reduced pressure. Purification by silica gel chromatography (CH₂Cl₂) afforded (S)-methyl 2-((4Z,7Z,10Z,13Z,16Z,19Z)-docosa-4,7,10,13,16,19-hexaenamido)propanoate.

(S)-methyl 2-((4Z,7Z,10Z,13Z,16Z,19Z)-docosa-4,7,10,13,16,19-hexaenamido)propanoate was taken up in THF (5 mL) along with 5M aqueous NaOH (3 mL) and the resulting reaction mixture was stirred at room temperature for 2 h. The mixture was concentrated under reduced pressure and the resulting residue was diluted with water and the pH was adjusted to 2 with 5M aqueous HCl. The resulting mixture was extracted with CH₂Cl₂ and the combined organic extracts were washed with brine, dried over Na₂SO₄, filtered and concentrated under reduced pressure to afford (S)-2-((4Z,7Z,10Z,13Z,16Z,19Z)-docosa-4,7,10,13,16,19-hexaenamido)propanoic acid.

(S)-2-((4Z,7Z,10Z,13Z,16Z,19Z)-Docosa-4,7,10,13,16,19-hexaenamido)propanoic acid (1 mmol) was taken up in CH₃CN (8 mL) along with L-threonine methyl ester (1 mmol), HATU (1.1 mmol) and DIEA (1.5 mmol). The resulting reaction mixture was stirred at room temperature for 5 h and diluted with EtOAc. The organic layer was washed with dilute aqueous NaHCO₃, brine, dried over Na₂SO₄, filtered and concentrated under reduced pressure. The resulting residue was purified by silica gel chromatography (CH₂Cl₂) to afford (2S,3R)-methyl 2-(tert-butoxycarbonyl)-3-(S)-2-((4Z,7Z,10Z,13Z,16Z,19Z)-docosa-4,7,10,13,16,19-hexaenamido)propanoyloxy)butanoate.

(2S,3R)-Methyl 2-(tert-butoxycarbonyl)-3-((S)-2-((4Z,7Z,10Z,13Z,16Z,19Z)-docosa-4,7,10,13,16,19-hexaenamido)propanoyloxy)butanoate (0.5 mmol) was taken up in 4M HCl in dioxane (2 mL). The resulting reaction mixture was allowed to stand at room temperature for 10 min, then diluted with EtOAc (10 mL) and concentrated under reduced pressure to afford the HCl salt of (2S,3R)-methyl 2-amino-3-((S)-2-((4Z,7Z,10Z,13Z,16Z,19Z)-docosa-4,7,10,13,16,19-hexaenamido)propanoyloxy)butanoate.

The HCl salt of (2S,3R)-methyl 2-amino-3-((S)-2-((4Z,7Z,10Z,13Z,16Z,19Z)-docosa-4,7,10,13,16,19-hexaenamido)propanoyloxy)butanoate was taken up in CH₃CN (5 mL) along with (5Z,8Z,11Z,14Z,17Z)-eicosa-5,8,11,14,17-pentaenoic acid (0.35 mmol), HATU (0.55 mmol) and DIEA (1.5 mmol). The resulting reaction mixture was stirred at room temperature for 1 h and concentrated under reduced pressure. The resulting residue is purified by silica gel chromatography to afford (2S,3R)-methyl 3-((S)-2-((4Z,7Z,10Z,13Z,16Z,19Z)-docosa-4,7,10,13,16,19-hexaenamido)propanoyloxy)-2-((5Z,8Z,11Z,14Z,17Z)-icosa-5,8,11,14,17-pentaenamido)butanoate. MS (EI) calcd for C₅₀H₇₄N₂O₆: 798.55. found 799 (M+1).

### Example 15

### Preparation of (S)-2-((4Z,7Z,10Z,13Z,16Z,19Z)-docosa-4,7,10,13,16,19-hexaenoylamino)-6-((5Z,8Z,11Z,14Z,17Z)-icosa-5,8,11,14,17-pentaenoylamino)-hexanoic acid (I-45)

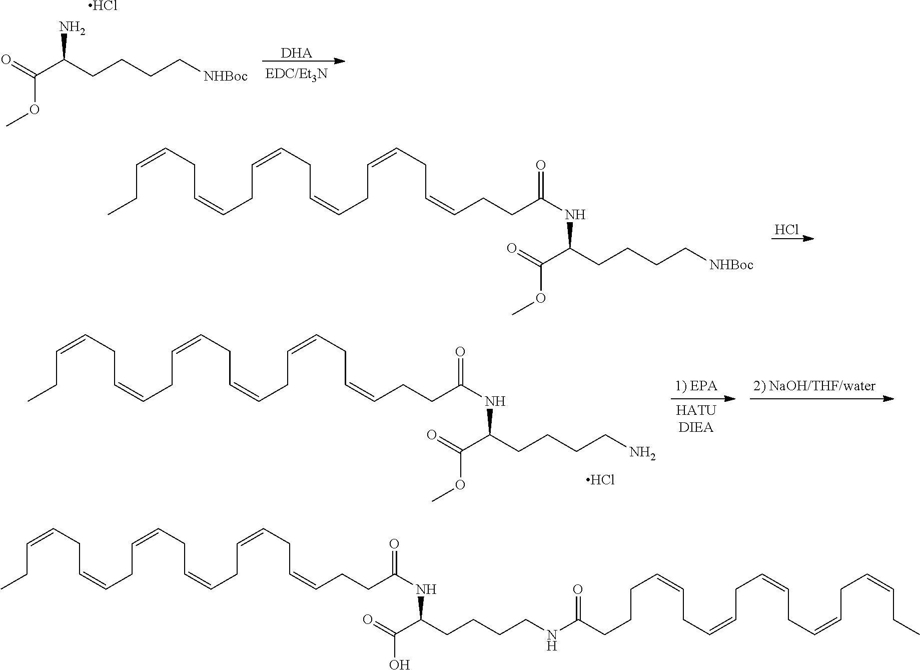

In a typical run, H-L-Lys(OMe). HCl (35 g, 0.118 mol) was taken up in 600 mL of CH₂Cl₂ along with DHA (38.7 g, 0.118 mol) along with EDC (25 g, 0.130 mol). The resulting reaction mixture was stirred at rt for 3 h and washed with saturated NH₄Cl. The organic layer was washed with brine, dried (Na₂SO₄) and concentrated under reduced pressure. Purification by chromatography (CH₂Cl₂) afforded 34 g of (S)-methyl 6-(tert-butoxycarbonyl)-244Z,7Z,10Z,13Z,16Z,19Z)-docosa-4,7,10,13,16,19-hexaenamido)hexanoate.

(S)-methyl 6-(tert-butoxycarbonyl)-2-((4Z,7Z,10Z,13Z,16Z,19Z)-docosa-4,7,10,13,16,19-hexaenamido)hexanoate (400 mg, 0.702 mmol) was taken up in 3 mL of 4 N HCl in dioxane and allowed to stir at rt for 10 min. The reaction mixture was diluted with 20 mL of EtOAc and concentrated under reduced pressure. The resulting product was used immediately for the next step. This HCl salt was taken up in 5 mL of CH₃CN along with EPA (212 mg, 0.702 mmol) HATU (293 mg, 0.77 mmol) and DIEA (370 μL, 2.1 mmol). The resulting reaction mixture was stirred at rt under N₂ for 2 h. It was then quenched with saturated NH₄Cl and concentrated. The aqueous mixture was extracted with EtOAc. The combined organic layers were washed with brine, dried (Na₂SO₄) and concentrated under reduced pressure. Chromatography (95% CH₂Cl₂. 5% MeOH) afforded 500 mg of (S)-methyl 2-((4Z,7Z,10Z,13Z,16Z,19Z)-docosa-4,7,10,13,16,19-hexaenamido)-6-((5Z,8Z,11Z,14Z,17Z)-icosa-5,8,11,14,17-pentaenamido)hexanoate.

(S)-methyl 2-((4Z,7Z,10Z,13Z,16Z,19Z)-docosa-4,7,10,13,16,19-hexaenamido)-6-((5Z,8Z,11Z,14Z,17Z)-icosa-5,8,11,14,17-pentaenamido)hexanoate (prepared above, 500 mg, 0.660 mmol) was taken up in 5 mL of THF and NaOH (80 mg, 2 mmol) was added as a solution in 3 mL of water. The resulting reaction mixture was stirred at rt for 1 h. It was then acidified to pH 3-4 with 1 N HCl and concentrated under reduced pressure. The aqueous layer was extracted with EtOAc (3×25 mL). The combined organic layers were washed with water (6×20 mL) with periodic check of the water layer to be sure that the pH is close to neutral. The organic layer was washed with brine, dried (Na₂SO₄) and concentrated under reduced pressure to afford 200 mg of (S)-2-((4Z,7Z,10Z,13Z,16Z,19Z)-docosa-4,7,10,13,16,19-hexaenoylamino)-6-((5Z,8Z,11Z,14Z,17Z)-icosa-5,8,11,14,17-pentaenoylamino)-hexanoic acid. MS (EI) calcd for C₄₈H₇₂N₂O₄: 740.55. found 741 (M+1).

### Example 16

### Preparation of (4Z,7Z,10Z,13Z,16Z,19Z)-1-[4-((5Z,8Z,11Z,14Z,17Z)-icosa-5,8,11,14,17-pentaenoyl)-piperazin-1-yl]-docosa-4,7,10,13,16,19-hexaen-1-one (I-67)

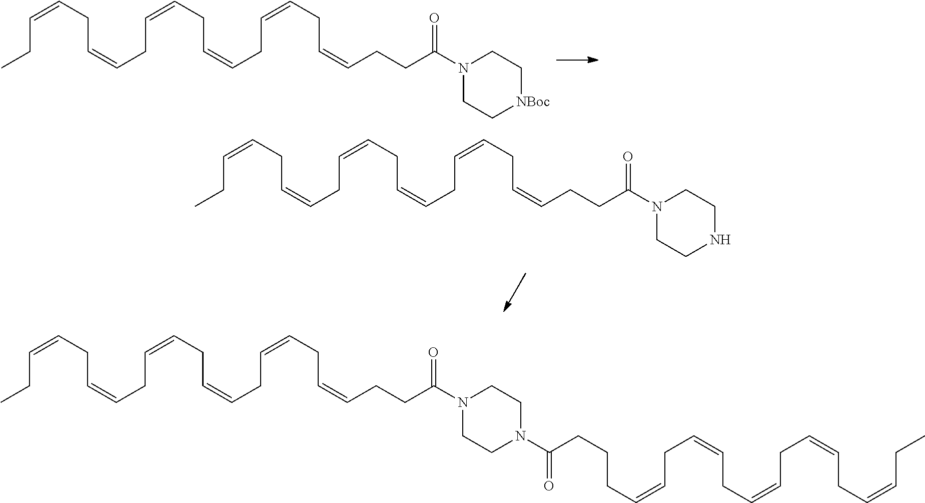

tert-Butyl 4-(4Z,7Z,10Z,13Z,16Z,19Z)-docosa-4,7,10,13,16,19-hexaenoylpiperazine-1-carboxylate was prepared using the same experimental procedure detailed above in the preparation of tert-butyl 2-(4Z,7Z,10Z,13Z,16Z,19Z)-docosa-4,7,10,13,16,19-hexaenamidoethylcarbamate. tert-Butyl 4-(4Z,7Z,10Z,13Z,16Z,19Z)-docosa-4,7,10,13,16,19-hexaenoylpiperazine-1-carboxylate (550 mg, 2.22 mmol) was taken up in 4 mL of 4 N HCl in dioxane and allowed to stir at room temperature for 10 min. The resulting reaction mixture was concentrated under reduced pressure to afford the HCl salt of (4Z,7Z,10Z,13Z,16Z,19Z)-1-(piperazin-1-yl)docosa-4,7,10,13,16,19-hexaen-1-one. This HCl salt of (4Z,7Z,10Z,13Z,16Z,19Z)-1-(piperazin-1-yl)docosa-4,7,10,13,16,19-hexaen-1-one (1.11 mmol) was taken up in 5 mL of CH₃CN along with EPA (335 mg, 1.11 mmol), HATU (365 mg, 1.22 mmol) and DIEA (580 μL, 3.33 mmol). The resulting reaction mixture was stirred at room temperature for 2 h and diluted with EtOAc (30 mL). The organic layer was washed with brine, dried (Na₂SO₄) and concentrated under reduced pressure. Purification by chromatography (95% CH₂Cl₂, 5% MeOH) afforded 600 mg of (4Z,7Z,10Z,13Z,16Z,19Z)-1-[4-((5Z,8Z,11Z,14Z,17Z)-icosa-5,8,11,14,17-pentaenoyl)-piperazin-1-yl]-docosa-4,7,10,13,16,19-hexaen-1-one. MS (EI) calcd for C₄₆H₆₈N₂O₂: 680.53. found 681 (M+1).

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A molecular conjugate, directly or indirectly covalently linked wherein the linker comprises at least one amide, comprising two or more fatty acids selected from the group consisting of omega-3 fatty acids, fatty acids that are metabolized in vivo to omega-3 fatty acids, and lipoic acid, with the proviso that the molecular conjugate is not (4Z,7Z,10Z,13Z,16Z,19Z)-docosa-4,7,10,13,16,19-hexaenoic acid {2-[2-((4Z,7Z,10Z,13Z,16Z,19Z)-docosa-4,7,10,13,16,19-hexaenoylamino)-ethylamino]-ethyl}-amide (A); (5Z,8Z,11Z,14Z,17Z)-icosa-5,8,11,14,17-pentaenoic acid {2-[2-((5Z,8Z,11Z,14Z,17Z)-icosa-5,8,11,14,17-pentaenoylamino)-ethylamino]-ethyl}-amide (B); (4Z,7Z,10Z,13Z,16Z,19Z)-docosa-4,7,10,13,16,19-hexaenoic acid {2-[2-((4Z,7Z,10Z,13Z,16Z,19Z)-docosa-4,7,10,13,16,19-hexaenoylamino)-ethoxy]-ethyl}-amide (C); (5Z,8Z,11Z,14Z,17Z)-icosa-5,8,11,14,17-pentaenoic acid {2-[2-((5Z,8Z,11Z,14Z,17Z)-icosa-5,8,11,14,17-pentaenoylamino)-ethoxy]-ethyl}-amide (D); or (S)-2,6-Bis-((4Z,7Z,10Z,13Z,16Z,19Z)-docosa-4,7,10,13,16,19-hexaenoylamino)-hexanoic acid (E).

2. The molecular conjugate of claim 1, wherein the fatty acid is selected from the group consisting of all-cis-7,10,13-hexadecatrienoic acid, α-linolenic acid, stearidonic acid, eicosatrienoic acid, eicosatetraenoic acid, eicosapentaenoic acid (EPA), docosapentaenoic acid, docosahexaenoic acid (DHA), tetracosapentaenoic acid, tetracosahexaenoic acid and lipoic acid.

3. The molecular conjugate of claim 2, wherein the fatty acid is selected from eicosapentaenoic acid, docosahexaenoic acid and lipoic acid.

4. A compound of the Formula I: 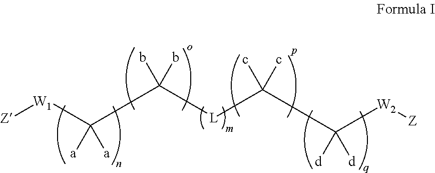
or a pharmaceutically acceptable salt, hydrate, solvate, prodrug, enantiomer or stereoisomer thereof;
wherein
W₁ and W₂ are each independently O, S, NH, NR, or W₁ and W₂ can be taken together can form an imidazolidine or piperazine group;
with the proviso that W₁ and W₂ can not simultaneously be 0 and one of W1 and W2 is NH or NR;
each a, b, c, and d is independently —H, -D, —CH₃, —OCH₃, —OCH₂CH₃, —C(O)OR, —O—Z, or benzyl, or two of a, b, c, and d can be taken together, along with the single carbon to which they are bound, to form a cycloalkyl or heterocycle;
each n, o, p, and q is independently 0, 1 or 2;
L is independently null, —O—, —S—, —S(O)—, —S(O)₂—, —S—S—, —(C₁-C₆alkyl)-, —(C₃-C₆cycloalkyl)-, a heterocycle, a heteroaryl, 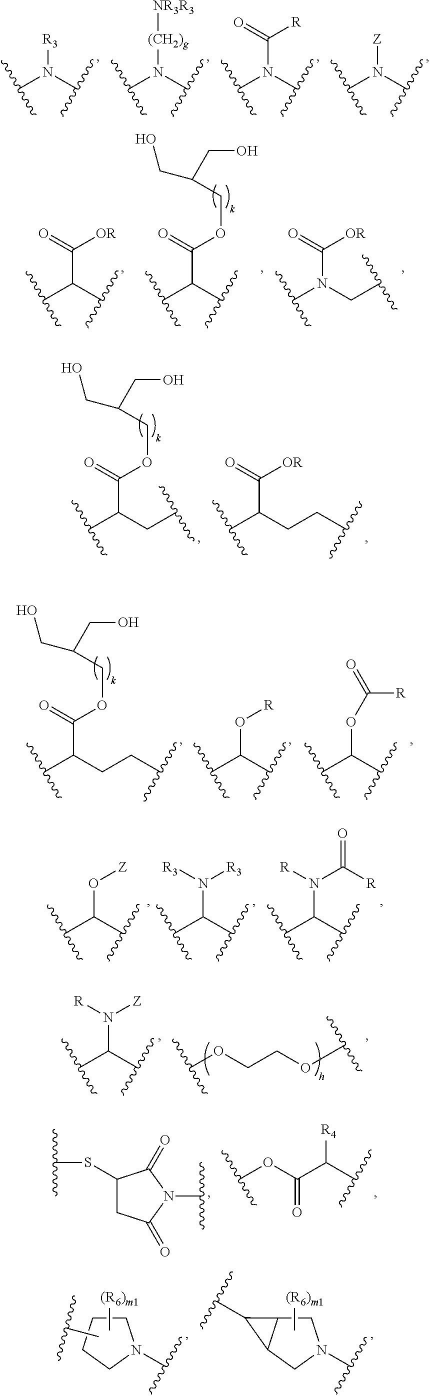 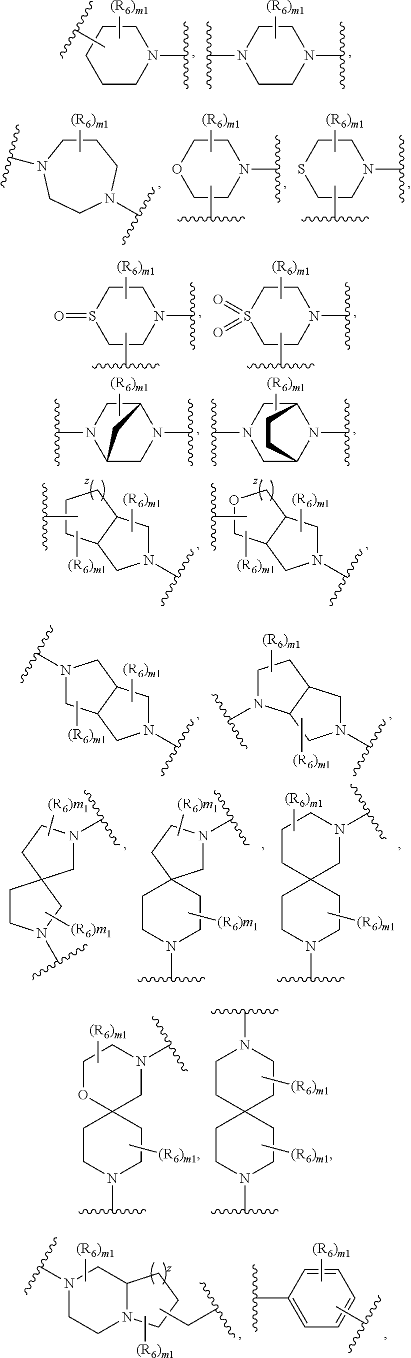
wherein the representation of L is not limited directionally left to right as is depicted, rather either the left side or the right side of L can be bound to the W₁ side of the compound of Formula I;
R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;
each g is independently 2, 3 or 4;
each h is independently 1, 2, 3 or 4;
m is 0, 1, 2, or 3; if m is more than 1, then L can be the same or different;
m1 is 0, 1, 2 or 3;
k is 0, 1, 2, or 3;
z is 1, 2, or 3;
each R₃ is independently H or C₁-C₆ alkyl that can be optionally substituted with either O or N and in NR₃R₃, both R₃ when taken together with the nitrogen to which they are attached can form a heterocyclic ring such as a pyrrolidine, piperidine, morpholine, piperazine or pyrrole;
each R₄ independently e, H or straight or branched C₁-C₁₀ alkyl which can be optionally substituted with OH, NH₂, CO₂R, CONH₂, phenyl, C₆H₄OH, imidazole or arginine;
each e is independently H or any one of the side chains of the naturally occurring amino acids;
each Z and Z′ is independently —H, 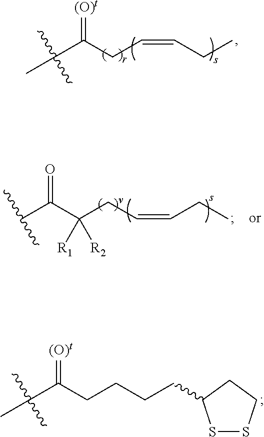
with the proviso that there is at least two of 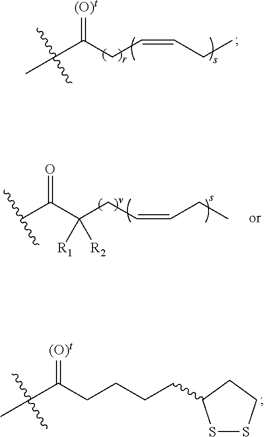
in the compound;
each r is independently 2, 3, or 7;
each s is independently 3, 5, or 6;
each t is independently 0 or 1;
each v is independently 1, 2, or 6;
R₁ and R₂ are independently —H, -D, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and
each R is independently —H, —C₁-C₃ alkyl, or straight or branched C₁-C₄ alkyl optionally substituted with OH, or halogen;
with the further proviso that the compound is not (4Z,7Z,10Z,13Z,16Z,19Z)-docosa-4,7,10,13,16,19-hexaenoic acid {2-[2-((4Z,7Z,10Z,13Z,16Z,19Z)-docosa-4,7,10,13,16,19-hexaenoylamino)-ethylamino]-ethyl}-amide (A); (5Z,8Z,11Z,14Z,17Z)-icosa-5,8,11,14,17-pentaenoic acid {2-[2-((5Z,8Z,11Z,14Z,17Z)-icosa-5,8,11,14,17-pentaenoylamino)-ethylamino]-ethyl}-amide (B); (4Z,7Z,10Z,13Z,16Z,19Z)-docosa-4,7,10,13,16,19-hexaenoic acid {2-[2-((4Z,7Z,10Z,13Z,16Z,19Z)-docosa-4,7,10,13,16,19-hexaenoylamino)-ethoxy]-ethyl}-amide (C); (5Z,8Z,11Z,14Z,17Z)-icosa-5,8,11,14,17-pentaenoic acid {2-[2-((5Z,8Z,11Z,14Z,17Z)-icosa-5,8,11,14,17-pentaenoylamino)-ethoxy]-ethyl}-amide (D); or (S)-2,6-Bis-((4Z,7Z,10Z,13Z,16Z,19Z)-docosa-4,7,10,13,16,19-hexaenoylamino)-hexanoic acid (E)

5. The compound of claim 4, wherein W₁ and W₂ are each NH.

6. The compound of claim 5, wherein each of n, o, p, and q is 1,

7. The compound of claim 5, wherein two of n, o, p, and q is each 1,

8. The compound of claim 6 or 7, wherein m is 1.

9. The compound of claim 8, wherein L is selected from —O—, —N(R₄)—,

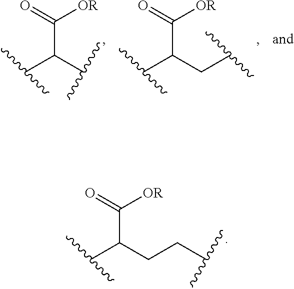

10. The compound of claim 4 wherein W₁ and W₂ are taken together to form a piperazine.

11. A compound of claim 4, wherein the compound is selected from a group consisting of
(4Z,7Z,10Z,13Z,16Z,19Z)-docosa-4,7,10,13,16,19-hexaenoic acid [2-((4Z,7Z,10Z,13Z,16Z,19Z)-docosa-4,7,10,13,16,19-hexaenoylamino)-ethyl]-amide (I-1);
(4Z,7Z,10Z,13Z,16Z,19Z)-docosa-4,7,10,13,16,19-hexaenoic acid (2-{[2-((4Z,7Z,10Z,13Z,16Z,19Z)-docosa-4,7,10,13,16,19-hexaenoylamino)-ethyl]-methyl-amino}-ethyl)-amide (I-2);
(S)-2,6-bis-((4Z,7Z,10Z,13Z,16Z,19Z)-docosa-4,7,10,13,16,19-hexaenoylamino)-hexanoic acid 2-hydroxy-1-hydroxymethyl-ethyl ester (I-5);
(4Z,7Z,10Z,13Z,16Z,19Z)-1-[4-((4Z,7Z,10Z,13Z,16Z,19Z)-docosa-4,7,10,13,16,19-hexaenoyl)-piperazin-1-yl]-docosa-4,7,10,13,16,19-hexaen-1-one (I-25);
(4Z,7Z,10Z,13Z,16Z,19Z)-docosa-4,7,10,13,16,19-hexaenoic acid [2-((5Z,8Z,11Z,14Z,17Z)-icosa-5,8,11,14,17-pentaenoylamino)-ethyl]-amide (I-37);
(4Z,7Z,10Z,13Z,16Z,19Z)-docosa-4,7,10,13,16,19-hexaenoic acid {2-[2-((5Z,8Z,11Z,14Z,17Z)-icosa-5,8,11,14,17-pentaenoylamino)-ethoxy]-ethyl}-amide (I-38);
(4Z,7Z,10Z,13Z,16Z,19Z)-docosa-4,7,10,13,16,19-hexaenoic acid (2-{[2((5Z,8Z,11Z,14Z,17Z)-icosa-5,8,11,14,17-pentaenoylamino)-ethyl]-methyl-amino}-ethyl)-amide (I-39);
(4Z,7Z,10Z,13Z,16Z,19Z)-docosa-4,7,10,13,16,19-hexaenoic acid {2-[2-((5Z,8Z,11Z,14Z,17Z)-icosa-5,8,11,14,17-pentaenoylamino)-ethylamino]-ethyl}-amide (I-40);
(S)-6-((4Z,7Z,10Z,13Z,16Z,19Z)-docosa-4,7,10,13,16,19-hexaenoylamino)-2-((5Z,8Z,11Z,14Z,17Z)-icosa-5,8,11,14,17-pentaenoylamino)-hexanoic acid (I-44);
(S)-2-((4Z,7Z,10Z,13Z,16Z,19Z)-docosa-4,7,10,13,16,19-hexaenoylamino)-6-((5Z,8Z,11Z,14Z,17Z)-icosa-5,8,11,14,17-pentaenoylamino)-hexanoic acid (I-45);
(S)-6-((4Z,7Z,10Z,13Z,16Z,19Z)-docosa-4,7,10,13,16,19-hexaenoylamino)-2-((5Z,8Z,11Z,14Z,17Z)-icosa-5,8,11,14,17-pentaenoylamino)-hexanoic acid 2-hydroxy-1-hydroxymethyl-ethyl ester (I-46);
(S)-2-((4Z,7Z,10Z,13Z,16Z,19Z)-docosa-4,7,10,13,16,19-hexaenoylamino)-6-((5Z,8Z,11Z,14Z,17Z)-icosa-5,8,11,14,17-pentaenoylamino)-hexanoic acid 2-hydroxy-1-hydroxymethyl-ethyl ester (I-47);
(4Z,7Z,10Z,13Z,16Z,19Z)-1-[4-((5Z,8Z,11Z,14Z,17Z)-icosa-5,8,11,14,17-pentaenoyl)-piperazin-1-yl]-docosa-4,7,10,13,16,19-hexaen-1-one (I-67);
(5Z,8Z,11Z,14Z,17Z)-icosa-5,8,11,14,17-pentaenoic acid [2-((5Z,8Z,11Z,14Z,17Z)-icosa-5,8,11,14,17-pentaenoylamino)-ethyl]-amide (I-81);
(S)-2,6-bis-((5Z,8Z,11Z,14Z,17Z)-icosa-5,8,11,14,17-pentaenoylamino)-hexanoic acid (I-85);
(S)-2,6-bis-((5Z,8Z,11Z,14Z,17Z)-icosa-5,8,11,14,17-pentaenoylamino)-hexanoic acid 2-hydroxy-1-hydroxymethyl-ethyl ester (I-86); and
(5Z,8Z,11Z,14Z,17Z)-1-[4-((5Z,8Z,11Z,14Z,17Z)-icosa-5,8,11,14,17-pentaenoyl)-piperazin-1-yl]-icosa-5,8,11,14,17-pentaen-1-one (I-91).

12. A pharmaceutical composition comprising a molecular conjugate of claim 1 and a pharmaceutically acceptable carrier.

13. A pharmaceutical composition comprising a compound of Formula I′: 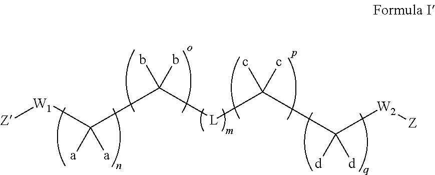
or a pharmaceutically acceptable salt, hydrate, solvate, prodrug, enantiomer or stereoisomer thereof;
wherein
W₁ and W₂ are each independently O, S, NH, NR, or W₁ and W₂ can be taken together can form an imidazolidine or piperazine group;
with the proviso that W₁ and W₂ can not simultaneously be 0 and one of W1 and W2 is NH or NR;
each a, b, c, and d is independently —H, -D, —CH₃, —OCH₃, —OCH₂CH₃, —C(O)OR, —O—Z, or benzyl, or two of a, b, c, and d can be taken together, along with the single carbon to which they are bound, to form a cycloalkyl or heterocycle;
each n, o, p, and q is independently 0, 1 or 2;
L is independently null, —O—, —S—, —S(O)—, —S(O)₂—, —S—S—, —(C₁-C₆alkyl)-, —(C₃-C₆cycloalkyl)-, a heterocycle, a heteroaryl, 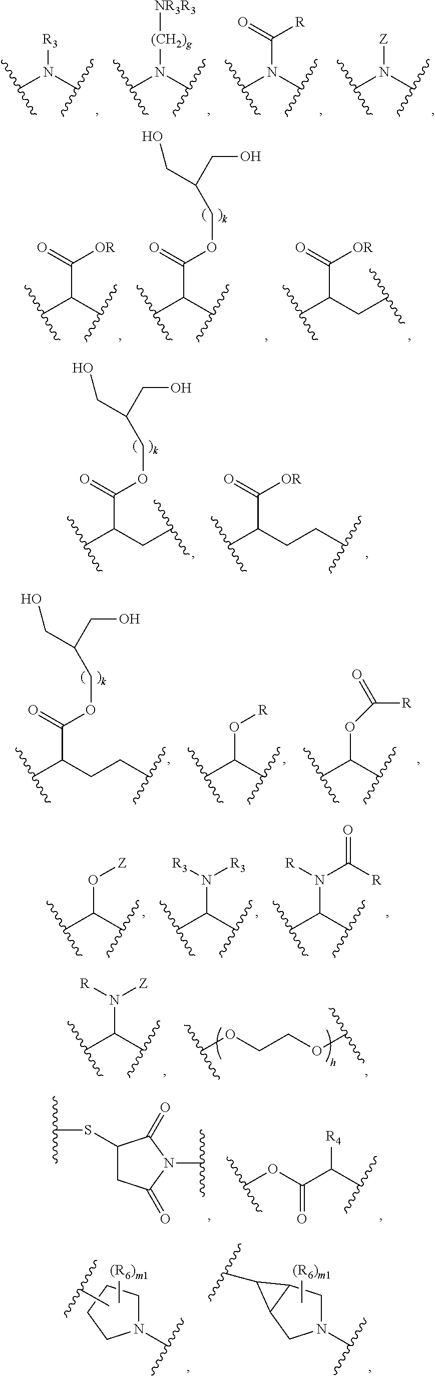 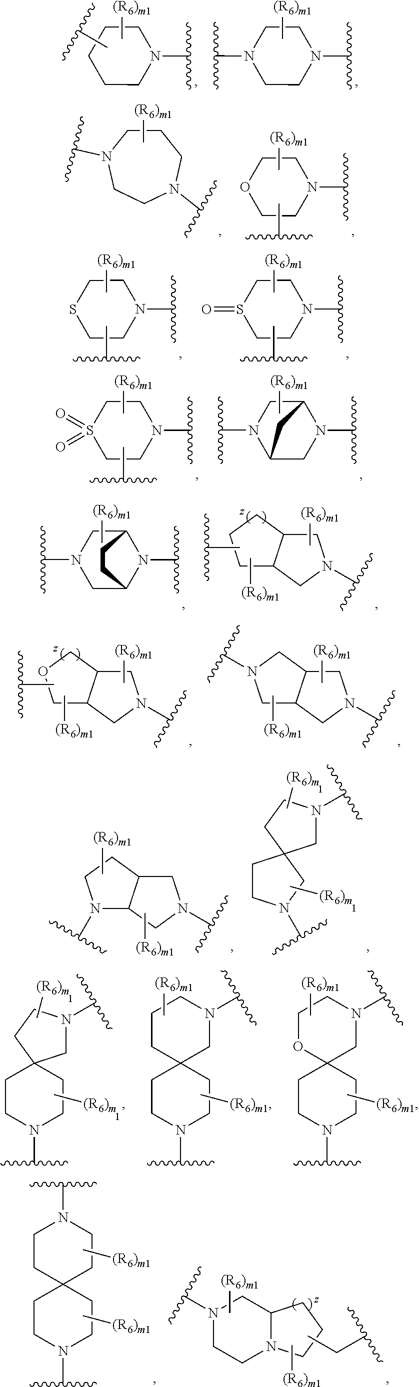 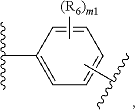
wherein the representation of L is not limited directionally left to right as is depicted, rather either the left side or the right side of L can be bound to the W₁ side of the compound of Formula I′;
R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;
each g is independently 2, 3 or 4;
each h is independently 1, 2, 3 or 4;
m is 0, 1, 2, or 3; if m is more than 1, then L can be the same or different;
m1 is 0, 1, 2 or 3;
k is 0, 1, 2, or 3;
z is 1, 2, or 3;
each R₃ is independently H or C₁-C₆ alkyl that can be optionally substituted with either O or N and in NR₃R₃, both R₃ when taken together with the nitrogen to which they are attached can form a heterocyclic ring such as a pyrrolidine, piperidine, morpholine, piperazine or pyrrole;
each R₄ independently e, H or straight or branched C₁-C₁₀ alkyl which can be optionally substituted with OH, NH₂, CO₂R, CONH₂, phenyl, C₆H₄OH, imidazole or arginine;
each e is independently H or any one of the side chains of the naturally occurring amino acids;
each Z and Z′ is independently —H, 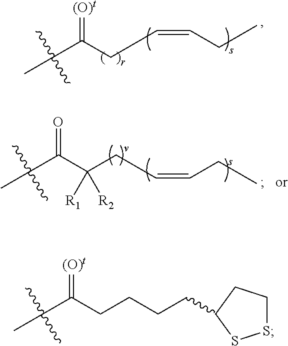
with the proviso that there is at least two of 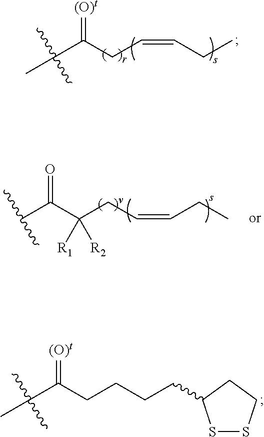
in the compound;
each r is independently 2, 3, or 7;
each s is independently 3, 5, or 6;
each t is independently 0 or 1;
each v is independently 1, 2, or 6;
R₁ and R₂ are independently —H, -D, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and
each R is independently —H, —C₁-C₃ alkyl, or straight or branched C₁-C₄ alkyl optionally substituted with OH, or halogen.
and a pharmaceutically acceptable carrier.

14. A method for treating a disease with inflammation as the underlying etiology, the method comprising administering to a patient in need thereof an effective amount of a pharmaceutical composition of claim 12.

15. The method of claim 14, wherein the disease is selected from metabolic disease, autoimmune disease, inflammatory respiratory disease, and neurodegenerative disease.

16. A method for treating a disease with inflammation as the underlying etiology, the method comprising administering to a patient in need thereof an effective amount of a pharmaceutical composition of claim 13.

17. The method of claim 16, wherein the disease is selected from metabolic disease, autoimmune disease, inflammatory respiratory disease, and neurodegenerative disease.

18. The method of claim 17, wherein the disease is metabolic disease.

19. The method of claim 18, wherein the metabolic disease is selected from atherosclerosis, dyslipidemia, coronary heart disease, hypertriglyceridemia, hypercholesterimia, Type 2 diabetes, elevated cholesterol, metabolic syndrome, diabetic nephropathy, progressive diabetic nephropathy, IgA nephropathy, chronic kidney disease (CKD) and cardiovascular disease, fatty liver disease, diabetic neuropathy, diabetic retinopathy, or metabolic syndrome.

20. The method of claim 19, wherein the disease is Type 2 diabetes.

21. The method of claim 19, wherein the disease is hypertriglyceridemia.

22. The method of claim 19, wherein the disease is IgA nephropathy.

23. The method of claim 17, wherein the disease is autoimmune disease.

24. The method of claim 23, wherein the autoimmune disease is selected from cystic fibrosis rheumatoid arthritis, psoriasis, systemic lupus erythematosus, and inflammatory bowel disease.

25. The method of claim 24, wherein the autoimmune disease is systemic lupus erythematosus.

26. The method of claim 24, wherein the autoimmune disease is cystic fibrosis.

27. The method of claim 24, wherein the autoimmune disease is inflammatory bowel disease.

28. The method of claim 27, wherein the inflammatory bowel disease is selected from ileitis, ulcerative colitis, Barrett's syndrome, and Crohn's disease.

29. The method of claim 17, wherein the disease is inflammatory lung disease.

30. The method of claim 29, wherein the inflammatory lung disease is selected from asthma, adult respiratory distress syndrome, chronic obstructive airway disease, COPD and cystic fibrosis.

31. The method of claim 17, wherein the disease is neurodegenerative disease.

32. The method of claim 31, wherein the neurodegenerative disease is selected from multiple sclerosis, Parkinson's disease, Alzheimer's disease, Huntington's disease, amyotrophic lateral sclerosis (ALS) and muscular dystrophy.

33. The method of claim 16, wherein the disease with inflammation as the underlying etiology is selected from inflammatory diseases of the kidney, uremic complications, glomerulonephritis and nephrosis; nephropathy, and microalbuminuria.
